(19) United States
(12) Patent Application Publication
(10) Pub. No.: US 2023/0129674 A1
(43) Pub. Date: Apr. 27, 2023

(54) EFFICIENT PREPARATION OF DOLASTATIN AND AURISTATIN ANALOGS THROUGH A COMMON INTERMEDIATE

(71) Applicant: Sigma-Aldrich Co. LLC, St. Louis, MO (US)

(72) Inventors: David Goeddel, Madison, WI (US); James Walker, Verona, WI (US); Emilia Groso, Madison, WI (US); Ross Bemowski, Madison, WI (US); Logan Berg, Madison, WI (US)

(21) Appl. No.: 17/905,907

(22) PCT Filed: Mar. 9, 2021
(86) PCT No.: PCT/US2021/021547

§ 371 Date: Sep. 8, 2022

Related U.S. Application Data
(60) Provisional application No. 62/987,150, filed on Mar. 9, 2020.

(51) Int. Cl. C07K 5/02; C07D 207/08; C07C 229/22; C07C 271/22

(52) U.S. Cl. CPC C07K 5/0205; C07D 207/08; C07C 229/22; C07C 271/22

(57) ABSTRACT

Methods for making a dolastatin, auristatin or related compounds comprising the steps of providing a universal dolastatin core of Formula (I) reacting the C-terminal carboxylic acid group with an amine (A) to form an amide bond and reacting the N-terminal amine with a carboxylic acid (CA) to form an amide bond, wherein the steps can be performed in either order. Also provided are an isolated salt of the universal dolastatin core for use in preparation of dolastatins, auristatins and related compounds. Also provided are a number of intermediates and process steps which are useful for the preparation of high purity dolastatin core and high purity dolastatin and auristatin compounds.

### CROSS-REFERENCE TO RELATED APPLICATION

This application claims the benefit of priority of U.S. Provisional Patent Application No. 62/987,150 filed Mar. 9, 2020, the entirety of which is incorporated herein by reference.

### BACKGROUND

Dolastatins and related Auristatins are classes of compounds recognized as important antineoplastic agents particularly when coupled to antibodies for delivery directly to cancerous cells. As with many complex natural products and derivatives, the syntheses of these molecules are generally quite intensive requiring numerous chemical steps. Today, scientists must develop each synthesis de novo and then require further development to enable cGMP manufacturing of dolastatin and auristatin-containing payloads for conjugates.

A need exists for new, more efficient methods of synthesis for production of these clinically important compounds and to enable development of new dolastatin, auristatins and related compounds.

### SUMMARY

Provided herein are methods for making dolastatins, auristatins or related compounds by starting with a compound of Formula I

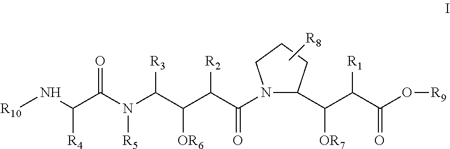

wherein R₁, R₂, R₃, R₄ R₅ and R₈ are each individually selected from H, C₁-C₆ alkyl, C₁-C₆ substituted alkyl, —OR₁₁, —NR₁₁R₁₂, —SR₁₁ and halo; R₁₁ and R₁₂ are individually selected from H, C₁-C₆ alkyl; R₆ and R₇ are each individually H or C₁-C₄ alkyl; R₉ is H or an acid protecting group, and R₁₀ is H or an amino protecting group, by reacting the C-terminal carboxylic acid group with an amine (A) to form an amide bond and reacting the N-terminal amine with a carboxylic acid (CA) to form an amide bond. The reaction steps, i.e., the reaction of the C-terminal carboxylic acid with an amine (A), or the reaction of the N-terminal amine with a carboxylic acid (CA), can be done in either order.

The amine (A) may be selected from alkylamines, alkanolamines, arylalkanolamines, amino acids, amino acid derivatives, peptides, and peptide derivatives. In various embodiments, the amine (A) may include one or more substituents. In some embodiments, the amine (A) includes a protecting group.

The carboxylic acid (CA) may be selected from amino acids, amino acid derivatives, peptides and peptide derivatives. In some embodiments, the carboxylic acid (CA) includes one or more substituents. In some embodiments, the carboxylic acid (CA) may have a protecting group.

In a preferred embodiment, the R groups in the compound of Formula I are selected to give the compound of Formula IA:

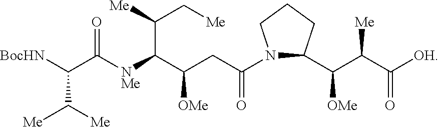

Also provided is an isolated salt of Formula II:

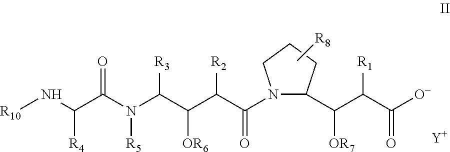

wherein R₁, R₂, R₃, R₄ R₅ and R₈ are each individually selected H, C₁-C₆ alkyl, C₁-C₆ substituted alkyl, —OR₁₁, —NR₁₁R₁₂, —SR₁₁ and halo, R₁₁ and R₁₂ are individually selected from H and C₁-C₆ alkyl, R₆ and R₇ are each individually H or C₁-C₄ alkyl, R₁₀ is H or an amino protecting group; and Y⁺ is counterion.

Also provided is a compound of Formula III:

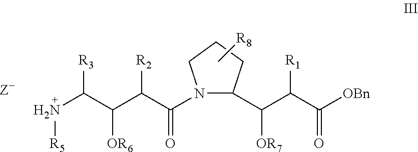

wherein R₁, R₂, R₃, R₅ and R₈ are each individually selected from H, C₁-C₆ alkyl, C₁-C₆ substituted alkyl, —OR₁₁, —NR₁₁R₁₂, —SR₁₁ and halo, R₁₁ and R₁₂ are individually selected from H and C₁-C₆ alkyl, R₆ and R₇ are each individually selected from H or C₁-C₄ alkyl, and Z⁻ is a counterion.

Also provided are additional intermediate compounds and process steps which are useful for the preparation of high purity dolastatin core and high purity dolastatin and auristatin compounds.

### BRIEF DESCRIPTION OF THE DRAWINGS

FIG. 1 shows the structural features of the preferred compound of Formula I.

FIG. 2 shows the synthetic scheme for the preparation of Formula I.

FIG. 3 shows exemplary dolastatin and auristatin payloads that have entered the clinic, including (A) MMAE, (B) MMAF, (C) XMT-1505, XMT-1536, (D) Amberstatin 269, (E) Auristatin W, (F) Dolastatin 10, and (G) Pfizer Auristatin, along with the universal dolastatin core described herein, the dolastatin core is highlighted in each payload.

FIG. 4 shows the synthetic schemes for the preparation of mc-Val-Cit-PAB-N-Me-Val-OH, MMAE and vcMMAE via two routes from Dolastatin Core.

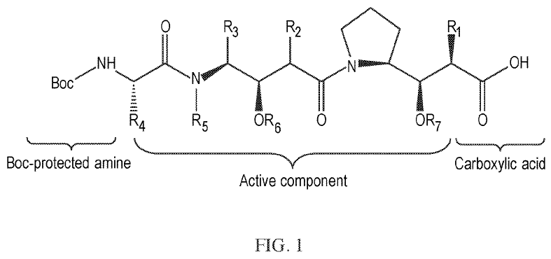

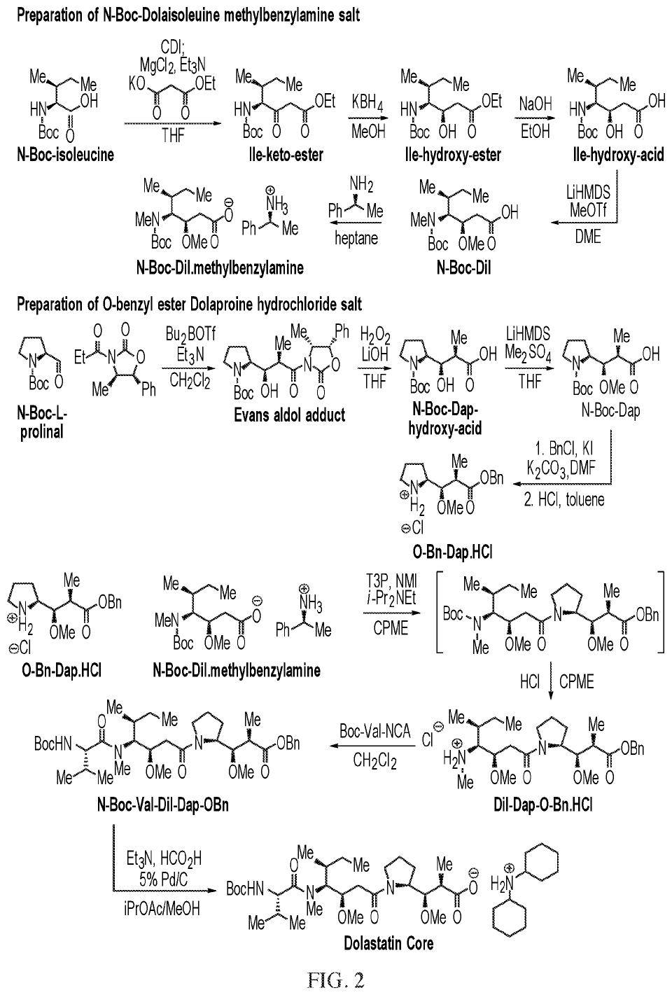

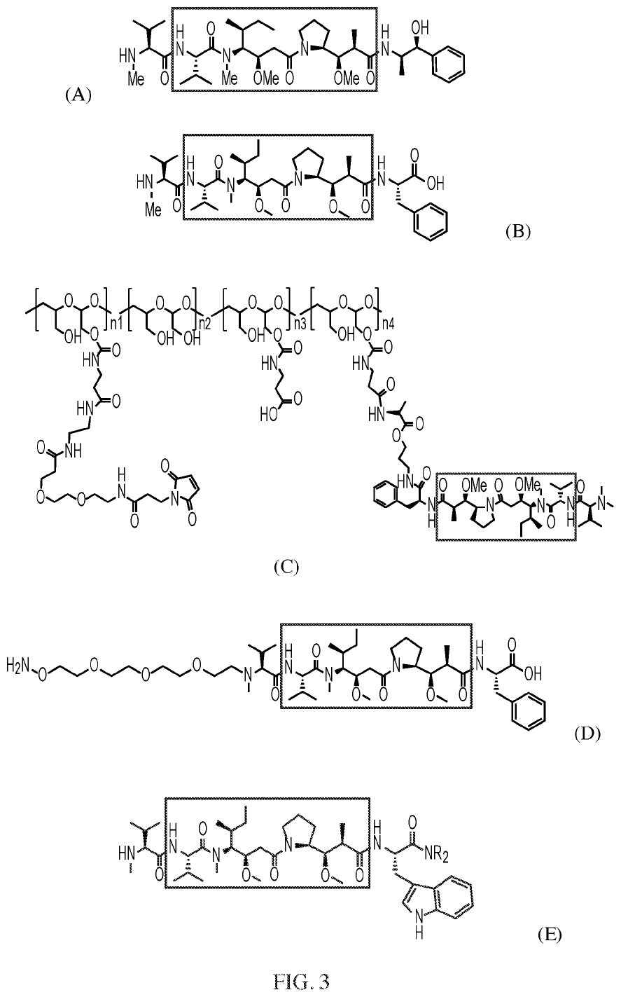

### DETAILED DESCRIPTION

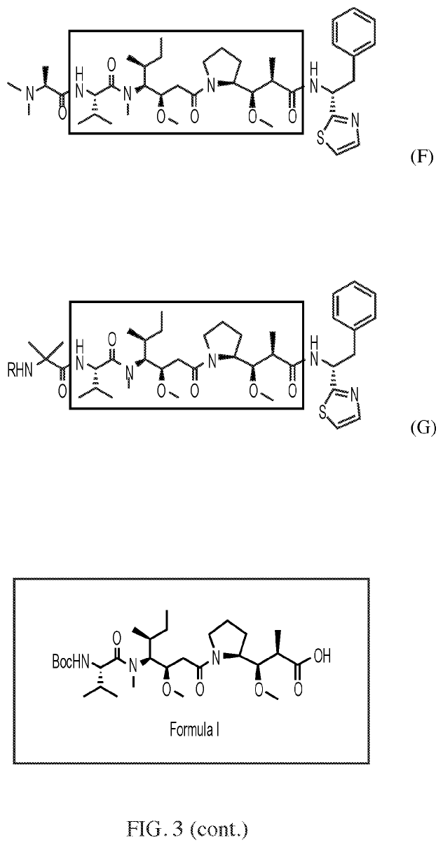

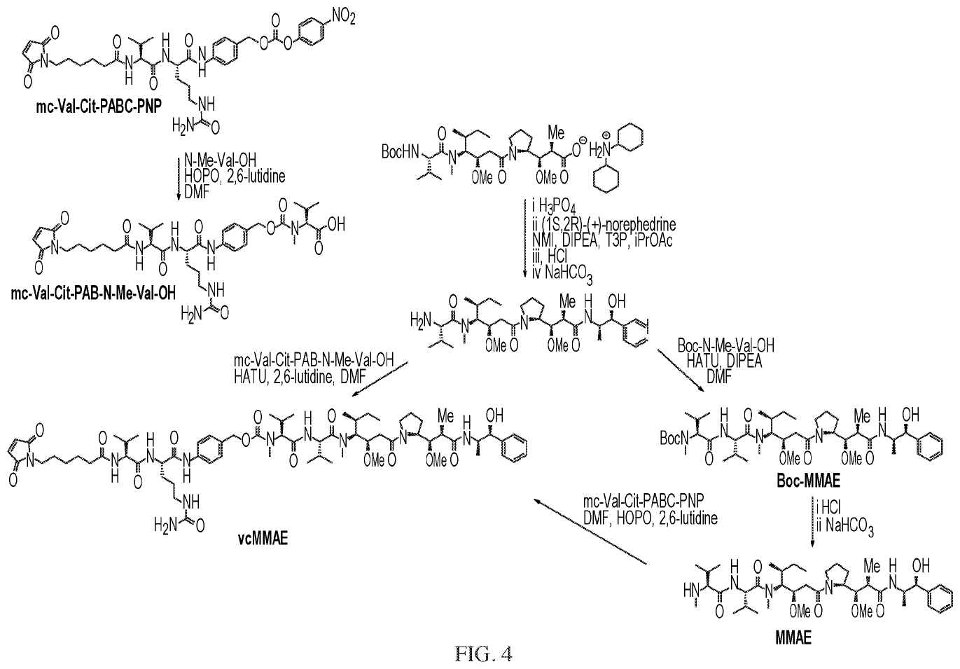

To overcome the deficiencies in the conventional methods of preparing auristatins and dolastatins, the inventors have identified and synthesized an advanced intermediate that can be efficiently transformed into numerous dolastatin, auristatin and related compounds. Through this identification and synthesis of a universal dolastatin core, the inventors have been able to develop a new platform that can be tailored to provide highly efficient syntheses of existing dolastatins and auristatins, such as those shown in FIG. 3. Additionally, this platform may be used to develop new dolastatins, auristatins and related compounds.

Provided herein are simplified methods for making dolastatins, auristatins or related compounds using a universal dolastatin core, shown in Formula I:

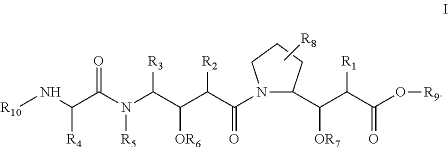

According to the methods provided herein, the C-terminal carboxylic acid is reacted with an amine (A) to form an amide bond, and the N-terminal amine is reacted with a carboxylic acid (CA) to form an amide bond, yielding a dolastatin, an auristatin or a further dolastatin or auristatin intermediate compound that can then be modified further, such as by the addition of one or more of a spacer, a linker, and an attachment group. These steps can be done in either order, that is, in some embodiments, the C-terminal carboxylic acid is first reacted with an amine (A), then the N-terminal amine is reacted with a carboxylic acid (CA). In other embodiments, the N-terminal amine is reacted with a carboxylic acid (CA) first, then C-terminal carboxylic acid is first reacted with an amine (A) to form the a dolastatin, an auristatin or a further dolastatin or auristatin intermediate of interest.

Suitable acid and amine protecting groups may be used to protect the terminal that is being reacted in a subsequent step. In embodiments in which there is a protecting group on the N-terminal amine, the protecting group can be removed by conventional methods prior to reacting with carboxylic acid (CA). When no protecting group is included, the optional deprotecting step is not necessary. Similarly, in embodiments in which there is a protecting group on the C-terminal carboxylic acid, the protecting group can be removed by conventional methods prior to reacting with amine (A). When no protecting group is included, the optional deprotecting step is not necessary.

Suitable coupling agents may be used may be used when reacting the C-terminal carboxylic acid with amine (A) and when reacting N-terminal amine with carboxylic acid (CA). Suitable coupling agents used in the methods provided herein include, but are not limited to, carbonyldiimidazole (CDI), propylphosphonic anhydride (T3P) solution and HATU. Other suitable coupling agents are known to those of ordinary skill in the art. In some embodiments, a coupling additive is used. Coupling additives are used in coupling reactions to inhibit side reaction and reduce racemization. Useful coupling additives for the reactions described herein include N-hydroxysuccinimide (HOSu), N-hydroxy-5-norbornene-2,3-dicarboximide (HONB), 1-hydroxybenzotriazole (HOBt), 6-chloro-1-hydroxybenzotriazole (6-Cl-HOBt), 1-hydroxy-7-azabenzotriazole (HOAt), 3-hydroxy-4-oxo-3,4-dihydro-1,2,3-benzotriazine (HODhbt), its aza derivative (HODhat), and 2-pyridinol 1-oxide (HOPO). Additionally, when reacting N-terminal amine with carboxylic acid (CA), the carboxylic acid may be in the form of a preformed activated ester. Common preformed activated esters include: N-hydroxysuccinimide (NHS-esters), 4-nitrophenol (PNP-ester), tetra/pentafluorophenol (TFP/PFP-esters) and N-carboxyanhydrides (NCA's). Other suitable preformed activated esters are known to those of ordinary skill in the art.

With respect to the universal dolastatin core of Formula I

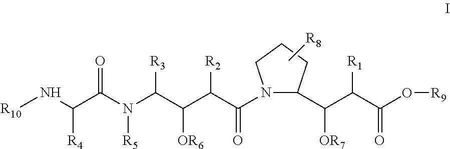

R₁, R₂, R₃, R₄ R₅ and R₈ are each individually selected from H, C₁-C₆ alkyl, C₁-C₆ substituted alkyl, —OR₁₁, —NR₁₁R₁₂, —SR₁₁ and halo; R₁₁ and R₁₂ are individually selected from H, C₁-C₆ alkyl; R₆ and R₇ are each individually H or C₁-C₄ alkyl; R₉ is H or an acid protecting group, and R₁₀ is H or an amino protecting group.

The amine (A) may be selected from alkylamines, alkanolamines, arylalkanolamines, amino acids, amino acid derivatives, peptides and peptide derivatives. When amine (A) is a peptide or peptide derivative, it is preferably 2-6 amino acid residues in length. In various embodiments, the amine (A) may include one or more substituents, suitable substituents include C₁-C₆ alkyl, hydroxy, C₁-C₆ alkoxy, amino, thiol, C₁-C₆ alkylthio, and halo. In some embodiments, amine (A) includes a protecting group. Suitable amine protecting groups include, but are not limited to, tert-butoxycarbonyl groups (Boc), 9-fluorenylmethoxycarbonyl groups (Fmoc), benzoyloxycarbonyl groups (Cbz, Z), and allyloxycarbonyl (Alloc). Suitable carboxyl protecting groups include, but are not limited to, simple esters, such as methyl ester, ethyl ester, tert-butyl ester, and benzyl ester, as well as esters formed with, e.g., trityl, 2,4-dimethoxylbenyl (Dmb), and 9-fluorenylmethyl (Fm). Other suitable protecting groups are known to those of ordinary skill in the art. In some embodiments, amine (A) may include one or more of a spacer, a linker and an attachment group.

In various embodiments, the amine (A) is selected from phenylalanine, phenylalanine derivatives, substituted phenylalanine, substituted phenylalanine derivatives, tryptophan, tryptophan derivatives, substituted tryptophan, substituted tryptophan derivatives, phenylpropanolamine, protected phenylpropanolamine, substituted phenylpropanolamine, protected substituted phenylpropanolamine, dolaphenine and protected dolaphenine, substituted dolaphenine, protected substituted dolaphenine, dolaphenine derivatives, protected dolaphenine derivatives, substituted dolaphenine derivatives, protected dolaphenine derivatives.

The carboxylic acid (CA) may be selected from amino acids, amino acid derivatives, peptides and peptide derivatives. When carboxylic acid (CA) is a peptide or peptide derivative, it is preferably 2-6 amino acid residues in length. In some embodiments, the carboxylic acid (CA) includes one or more substituents, suitable substituents include C₁-C₆ alkyl, hydroxy, C₁-C₆ alkoxy, amino, thiol, C₁-C₆ alkylthio, and halo. In some embodiments, the carboxylic acid (CA) may have a protecting group. Suitable carboxyl protecting groups include, but are not limited to, simple esters, such as methyl ester, ethyl ester, tert-butyl ester, and benzyl ester, as well as esters formed with, e.g., trityl, 2,4-dimethoxylbenyl (Dmb), and 9-fluorenylmethyl (Fm). Suitable amine protecting groups include, but are not limited to, tert-butoxycarbonyl groups (Boc), 9-fluorenylmethoxycarbonyl groups (Fmoc), benzoyloxycarbonyl groups (Cbz, Z), and allyloxycarbonyl (Alloc). Other suitable protecting groups are known to those of ordinary skill in the art. In some embodiments, carboxylic acid (CA) may include one or more of a spacer, a linker and an attachment group.

In various embodiments, carboxylic acid (CA) is selected from valine, protected valine, substituted valine, protected substituted valine, valine derivatives, protected valine derivatives, substituted valine derivatives, protected substituted valine derivatives, alanine, protected alanine, substituted alanine, protected substituted alanine, alanine derivatives, protected alanine derivatives, substituted alanine derivatives and protected substituted alanine derivatives.

In some preferred embodiments, the compound of Formula I is

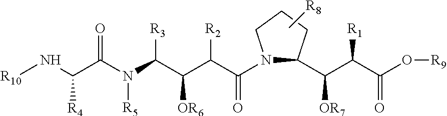

with the R groups as defined above.

In some preferred embodiments, the R groups in the compound of Formula I are selected to give the compound of Formula IA:

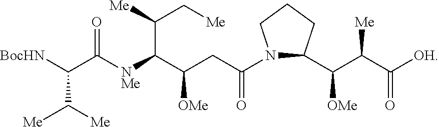

Also provided is an isolated salt of Formula II:

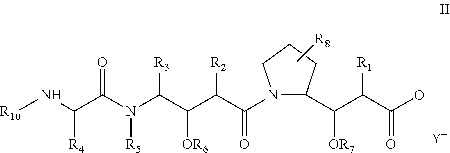

wherein R₁, R₂, R₃, R₄ R₅ and R₈ are each individually selected H, C₁-C₆ alkyl, C₁-C₆ substituted alkyl, —OR₁₁, —NR₁₁R₁₂, —SR₁₁ and halo, R₁₁ and R₁₂ are individually selected from H and C₁-C₆ alkyl, R₆ and R₇ are each individually H or C₁-C₄ alkyl, R₁₀ is H or an amino protecting group; and Y⁺ is counterion.

In some embodiments of the isolated salt of Formula II, R₁, R₂, R₃, R₄ R₅ and R₈ are each individually selected from H, methyl, ethyl, n-propyl, iso-propyl, n-butyl, sec-butyl, tert-butyl, and iso-butyl; R₆ and R₇ are each individually H or methyl, R₁₀ is H or tert-butoxycarbonyl (Boc), and is an ammonium ion of the formula N⁺HR₁₃R₁₄R₁₅ wherein R₁₃ is selected from optionally substituted C₁-C₈ alkyl and optionally substituted C₃-C₈ cycloalkyl; R₁₄ and R₁₅ are independently selected from H, optionally substituted C₁-C₈ alkyl and optionally substituted C₃-C₈ cycloalkyl; wherein each optional substituent, if present, is selected from alkyl and aryl.

The isolated salt according either of claims 10 or 11 wherein Y⁺ is selected from the group consisting diethylammonium ion, dibutylammonium ion, dicyclohexylammonium ion, methylcyclohexylammonium ion and methylbenzylammonium ion.

In some embodiments, the isolated salt of Formula II has the structure

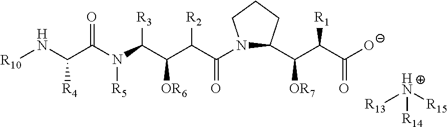

wherein the R groups are defined as above.

In a preferred embodiment, the isolated salt of Formula II has the structure

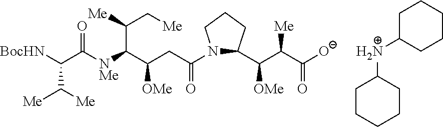

Also provided are additional compounds useful both in the preparation of the universal dolastatin core described herein as well as in alternate methods of preparing dolastatins, auristatins and related compounds. These compounds are intermediates in the preparation of the universal dolastatin core described herein. The use of these intermediates yields high purity dolastatin core as well as high purity dolastatin and auristatin compounds. It has also been found that the usefulness of these compounds in producing high purity compounds extends beyond the synthesis of the universal dolastatin core; they are also suitable in other methods of producing high purity dolastatin and auristatin compounds through other pathways.

Provided herein is a compound of Formula III:

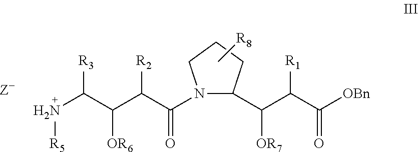

wherein R₁, R₂, R₃, R₅ and R₈ are each individually selected from H, C₁-C₆ alkyl, C₁-C₆ substituted alkyl, —OR₁₁, —NR₁₁R₁₂, —SR₁₁ and halo, R₁₁ and R₁₂ are individually selected from H and C₁-C₆ alkyl, R₆ and R₇ are each individually selected from H or C₁-C₄ alkyl, and Z⁻ is a counterion. In some embodiments, the compound of Formula III is in solution. In other embodiments, the compound of Formula III may be isolated. The salt form enables facile isolation and purification at scale. The crystallization enables impurity purge beyond the capabilities of column chromatography. The salt is a high purity, bench stable solid, whereas the free base is an oil. Using this salt will enable significantly larger scale production compared with methods requiring column chromatography.

In some embodiments of Formula III, R₁, R₂, R₃, R₅ and R₈ are each individually selected from H, methyl, ethyl, n-propyl, isopropyl, n-butyl, sec-butyl, iso-butyl, and tert-butyl; R₆ and R₇ are each individually selected from H, methyl, ethyl, n-propyl, isopropyl, n-butyl, sec-butyl, iso-butyl, and tert-butyl; and Z⁻ is selected from halide, sulfate, hydrogen sulfate, phosphate, hydrogen phosphate, dihydrogen phosphate, mesylate, tosylate, benzene sulfonate, ethylsulfonate, nitrate, formate, acetate, trifluoroacetate, oxalate, and citrate.

In some embodiments, the compound of Formula III is

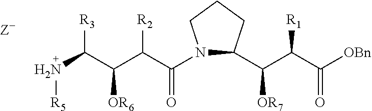

wherein the R groups and Z⁻ are defined as above.

In a preferred embodiment, the compound is of Formula III is:

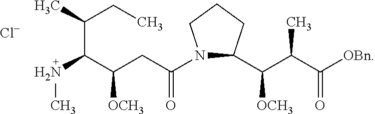 [SMILES: [Cl-] ; CC[C@H](C)[C@H](NC)[C@@H](CC(=O)N1CCC[C@H]1[C@H](OC)[C@@H](C)C(=O)OCc1ccccc1)OC]

Also provided is a method of coupling an amino acid to a compound of Formula III

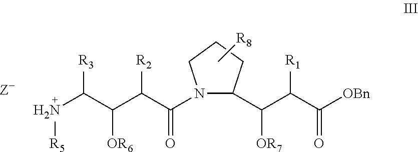

wherein R₁, R₂, R₃, R₅ and R₈ are each individually selected from H, C₁-C₆ alkyl, C₁-C₆ substituted alkyl, —OR₁₁, —NR₁₁R₁₂, —SR₁₁ and halo; R₁₁ and R₁₂ are individually selected from H and C₁-C₆ alkyl; R₆ and R₇ are each individually selected from H or C₁-C₄ alkyl, and Z⁻ is a counterion; by first contacting the compound of Formula III with an aqueous base to remove the counterion, and then contacting the compound of Formula III with an N-protected amino acid N-carboxyanhydride to yield a compound of Formula IV:

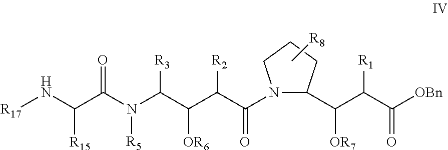

wherein R₁, R₂, R₃, R₅, R₆, R₇ and R₈ are defined as above; R₁₆ is an amino acid side chain, and R₁₇ is a protecting group. In various embodiments, the aqueous base is selected from Na₂CO₃, NaHCO₃, NaOH, Na₂HPO₄, and Na₃PO₄. The Boc-NCA coupling is operationally convenient and facilitates isolation of compounds of Formula IV, the reaction goes to completion and side products are easily purged by aqueous workup. Less than 0.5% epimerization is observed using Boc-NCA compared to 5-10% for HATU-mediated coupling. This is particularly noteworthy as HATU is a preferred coupling agent to minimize epimerization. This is particularly important because diastereomeric impurities are difficult to remove.

In some embodiments, of this method, the starting compound III, and final compound, IV, have R₁, R₂, R₃, R₅ and R₈ each individually selected from H, methyl, ethyl, n-propyl, isopropyl, n-butyl, sec-butyl, iso-butyl, and tert-butyl; R₆ and R₇ are each individually selected from H, methyl, ethyl, n-propyl, isopropyl, n-butyl, sec-butyl, iso-butyl, and tert-butyl, and Z⁻ is selected from halide, sulfate, hydrogen sulfate, phosphate, hydrogen phosphate, dihydrogen phosphate, mesylate, tosylate, benzene sulfonate, ethylsulfonate, nitrate, formate, acetate, trifluoroacetate, oxalate, and citrate, and the N-protected amino acid N-carboxyanhydride is a Boc-protected amino acid N-carboxyanhydride, and the the aqueous base is selected from Na₂CO₃, NaHCO₃, NaOH, Na₂HPO₄, and Na₃PO_{4′} and is preferably Na₂CO₃.

In still other embodiments, the compound of Formula III is

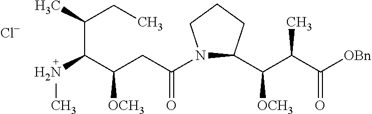 [SMILES: [Cl-] ; CC[C@H](C)[C@H](NC)[C@@H](CC(=O)N1CCC[C@H]1[C@H](OC)[C@@H](C)C(=O)OCc1ccccc1)OC]

the aqueous base is Na₂CO₃, the N-protected amino acid N-carboxyanhydride is Boc-Val-NCA, and the compound of Formula IV is:

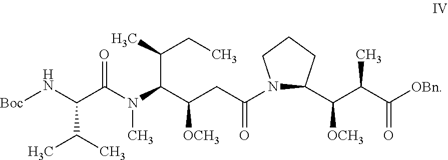 [SMILES: CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)OC(C)(C)C)C(C)C]

Also provided is a method of preparing a crystalline compound of Formula V without using column chromatography, the method involving the steps of providing a crude hydroxy acid of Formula V

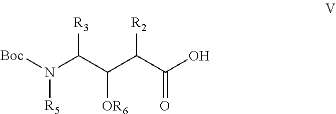

wherein R₂, R₃, and R₅ are each individually selected from H, C₁-C₆ alkyl, C₁-C₆ substituted alkyl, —OR₁₁, —NR₁₁R₁₂, —SR₁₁ and halo; R₁₁ and R₁₂ are individually selected from H, C₁-C₆ alkyl, and R₆ is selected from H and C₁-C₄ alkyl; dissolving crude hydroxy acid in a soluble solvent, adding an insoluble solvent, initiating crystallization, and allowing the crystallization to complete yielding a purified hydroxy acid of Formula V. The “soluble solvent” is a solvent in which the hydroxy acid is soluble in; in some embodiments, the soluble solvent is heated when dissolving the hydroxy acid. The “insoluble solvent” is a solvent in which the hydroxy acid is insoluble. In a preferred embodiment, the soluble solvent is tert-butyl methyl ether (MTBE) and the insoluble solvent is heptane. In some embodiments, crystallization is initiated by seeding the hydroxy acid. In other embodiments, crystallization is initiated by heat cycling. In still other embodiments, crystallization may be initiated by a combination of seeding and heat cycling. The resulting purified hydroxy acid may be isolated as a crystalline solid and is ready to use without additional purification—that is, no column chromatography is necessary when using this process. Advantageously, crystallization allows complete control of diastereoselectivity improving the quality and purity of downstream materials.

In some embodiments, the hydroxy acid of Formula V is

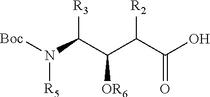

wherein R₂, R₃ and R₅ are each individually selected from the group consisting of H, methyl, ethyl, n-propyl, isopropyl, n-butyl, sec-butyl, iso-butyl, and tert-butyl; R₆ is selected from the group consisting of H and methyl. In a preferred embodiment, the soluble solvent is hot MTBE and the insoluble solvent is heptane. In various embodiments, crystallization may be initiated by seeding, by heat cycling or a combination of seeding and heat cycling.

In a preferred embodiment, the hydroxy acid of Formula V is

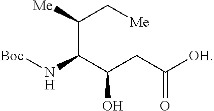 [SMILES: CC[C@H](C)[C@H](NC(=O)OC(C)(C)C)[C@H](O)CC(=O)O]

In some embodiments, the method further includes a step of isolating the purified hydroxy acid of Formula V. The resulting purified hydroxy acid of formula V is a crystalline solid.

In some embodiments, the method includes the synthesis of the hydroxy acid of Formula V without the need for column chromatography at any step. In an exemplary embodiment, the Ile-hydroxy acid shown above is synthesized starting with N-Boc isoleucine. The method involves condensing the N-Boc-isoleucine with mono-ethyl malonate to provide an Ile-keto-ester, reducing the Ile-keto-ester to provide an Ile-hydroxy-ester, and saponifying the Ile-hydroxy-ester to form a Ile-hydroxy-acid. The resulting Ile-hydroxy-acid is then purified by dissolving the crude Ile-hydroxy acid in a soluble solvent, adding an insoluble solvent, seeding the hydroxy acid to initiate crystallization, and allowing the crystallization to complete, yielding purified Ile-hydroxy-acid. The purified Ile-hydroxy acid is optionally filtered and dried to yield a crystalline solid.

Advantageously, this method provides a highly scalable method of preparing a compound of Formula V in high diastereoselectivity without the need for column chromatography. The crystallization step eliminates the need for column chromatography and provides the material in a crystalline form that can be stored for subsequent use.

Also provided is compound of Formula VI:

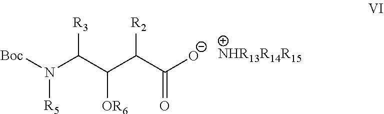

wherein R₂, R₃, and R₅ are each individually selected from H, C₁-C₆ alkyl, C₁-C₆ substituted alkyl, —OR₁₁, —NR₁₁R₁₂, —SR₁₁ and halo; R₁₁ and R₁₂ are individually selected from H, C₁-C₆ alkyl, and R₆ is selected from H and C₁-C₄ alkyl; and R₁₃ is selected from optionally substituted C₁-C₈ alkyl and optionally substituted C₃-C₈ cycloalkyl; R₁₄ and R₁₅ are independently selected from H, optionally substituted C₁-C₈ alkyl and optionally substituted C₃-C₈ cycloalkyl; wherein each optional substituent, if present, is selected alkyl and aryl. The salt form enables facile isolation and purification at scale. The crystallization enables impurity purge beyond the capability of column chromatography. The salt is a high purity, bench stable solid, whereas the free acid is an oil.

In some embodiment, the compound Formula VI is

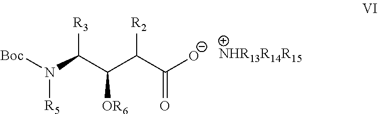

wherein R₂, R₃ and R₅ are each individually selected from H, methyl, ethyl, n-propyl, isopropyl, n-butyl, sec-butyl, iso-butyl, and tert-butyl, R₆ is selected from H and methyl; and NHR₁₃R₁₄R₁₅ is selected from diethylammonium ion, dibutylammonium ion, dicyclohexylammonium ion, methylcyclohexylammonium ion and methylbenzylammonium ion.

In still other embodiments, the compound of Formula VI is:

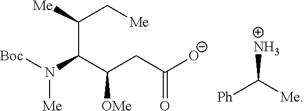 [SMILES: CC[C@H](C)[C@@H]([C@@H](CC(=O)[O-])OC)N(C)C(=O)OC(C)(C)C ; C[C@H]([NH3+])c1ccccc1]

Advantageously, the compound of formula VI may be isolated as a solid. In some embodiments, the compound of Formula VI is a crystalline solid.

Further provided is a compound of Formula VII

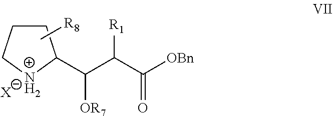

wherein R₁ and R₈ are individually selected from H, C₁-C₆ alkyl, C₁-C₆ substituted alkyl, —OR₁₁, —NR₁₁R₁₂, —SR₁₁ and halo; R₁₁ and R₁₂ are individually selected from H, C₁-C₆ alkyl; R₇ is selected from H or C₁-C₄ alkyl; and X⁻ is selected from halide, sulfate, hydrogen sulfate, phosphate, hydrogen phosphate, dihydrogen phosphate, mesylate, tosylate, benzene sulfonate, ethylsulfonate, nitrate, formate, acetate, oxalate, and citrate. Salt formation is advantageous as this material is unstable as the free base. The physical characteristics of the salt depends on the counterion. In some salt forms the material is a high purity, bench stable solid that when crystallized efficiently purges impurities. Other salt forms generate a viscous oil.

In some embodiments, the compound of Formula VII is

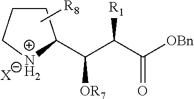

wherein R₁ and R₈ are individually selected from H, methyl, ethyl, n-propyl, isopropyl, n-butyl, sec-butyl, iso-butyl, and tert-butyl, R₇ is selected from H and methyl; and X⁻ is a halide, preferably chloride.

In still other embodiments, the Formula VII is

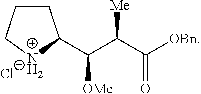 [SMILES: [Cl-] ; CO[C@@H]([C@@H]1CCC[NH2+]1)[C@@H](C)C(=O)OCc1ccccc1]

which is a high purity, bench stable solid that when crystallized efficiently purges impurities.

Advantageously, the compound of formula VII may be isolated as a solid. In some embodiments, the compound of Formula VII is a crystalline solid.

Also provided is a compound of Formula VIII:

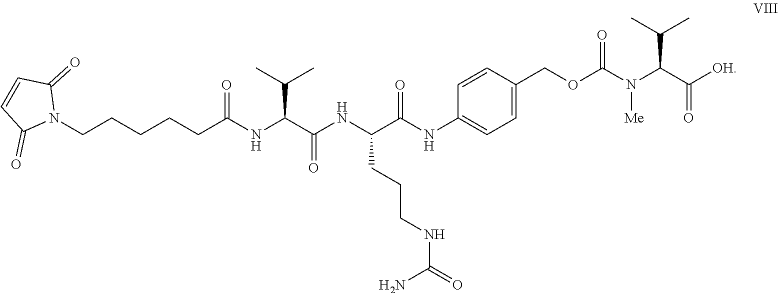 [SMILES: CC(C)[C@H](NC(=O)CCCCCN1C(=O)C=CC1=O)C(=O)N[C@@H](CCCNC(N)=O)C(=O)Nc1ccc(COC(=O)N(C)[C@H](C(=O)O)C(C)C)cc1]

This compound can advantageously be used in a simplified preparation of vcMMAE as well as other auristatins and dolastatins.

Also provided are methods of preparing specific auristatins of interest.

Also provided is a method of preparing monomethyl auristatin E from a core compound of Formula IA:

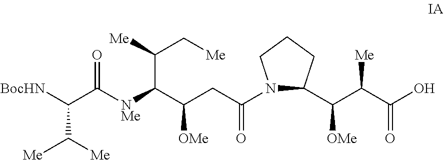

by contacting the core compound with norephedrine in the presence of a coupling agent to form a core compound-norephedrine intermediate, deprotecting the core compound-norephedrine intermediate to form a deprotected core compound-norephedrine intermediate, contacting the deprotected core compound-norephedrine intermediate with N-Boc-N-Me-Val-OH in the presence of a coupling agent to form N-Boc-MMAE, and deprotecting the N-Boc-MMAE to yield MMAE.

MMAE may then be used to prepare vcMMAE. For example, MMAE is contacted with mc-Val-Cit-PABC-PNP in the presence of a coupling additive. The reaction is allowed to proceed to completion and the product is purified by rp-HPLC.

Further provided is a method of preparing vc-MMAE from a core compound of Formula IA:

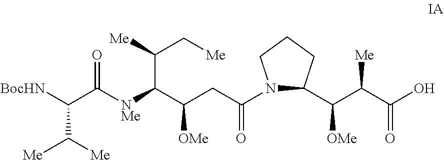

by contacting the core compound with norephedrine in the presence of a coupling agent to form a core compound-norephedrine intermediate, deprotecting the core compound-norephedrine intermediate to form a deprotected core compound-norephedrine intermediate, contacting the deprotected core compound-norephedrine intermediate with mc-Val-Cit-PAB-N-Me-Val-OH in the presence of a coupling agent to yield vcMMAE.

Also provided is a method of preparing monomethyl auristatin F from a core compound of Formula IA:

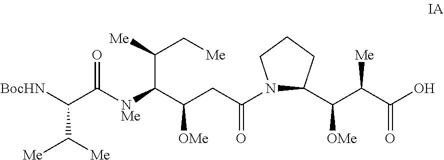

by contacting the core compound with L-phenylalanine methyl ester hydrochloride in the presence of a coupling agent to form N-Boc-Val-Dil-Dap-Phe-OMe, deprotecting the N-Boc-Val-Dil-Dap-Phe-OMe to yield Val-Dil-Dap-Phe-OMe, contacting Val-Dil-Dap-Phe-OMe with N-Boc-Me-Val-OH in the presence of a coupling agent to form N-Boc-N-Me-Val-Val-Dil-Dap-Phe-OMe, and deprotecting the N-Boc-N-Me-Val-Val-Dil-Dap-Phe-OMe to yield MMAF.

Other auristatins, dolastatins and related compounds may also be made using methods analogous to those described herein.

As used herein, the term “alkyl” refers to a straight chain or branched, saturated hydrocarbon. Representative alkyl groups include, but are not limited to, -methyl, -ethyl, -n-propyl,-n-butyl, -n-pentyl, -n-hexyl and so forth; exemplary branched alkyls include, but are not limited to, -isopropyl, -sec-butyl, -isobutyl, -tert-butyl, -isopentyl and 2-methylbutyl. An alkyl group may be attached at any available point to produce a stable compound. The term alkyl is also meant to encompass a fully substituted carbon.

The term “amino” refers to —NH₂, as well as “disubstituted amino” wherein one of the hydrogen atoms is replaced by a non-hydrogen substituent; and “trisubstituted amino” wherein both of the hydrogen atoms are replaced by non-hydrogen substituents, which may be identical or different.

The term “amine (A)” refers specifically to the amine-containing compound that is reacted with the C-terminal carboxylic acid of the universal dolastatin core described herein. The amine (A) may be selected from alkylamines, alkanolamines, arylalkanolamines, amino acids, amino acid derivatives, and peptides. In various embodiments, the amine (A) may include one or more substituents, suitable substituents include C₁-C₆ alkyl, hydroxy, C₁-C₆ alkoxy, amino, thiol, C₁-C₆ alkylthio, and halo. In some embodiments, amine (A) includes a protecting group. The protecting group may be an amine protecting group, a carboxyl protecting group, or a protecting group on the side chain or other protectable location. Suitable amine protecting groups include, but are not limited to, tert-butoxycarbonyl groups (Boc). 9-fluorenylmethoxycarbonyl groups (Fmoc), benzoyloxycarbonyl groups (Cbz, Z), and Allyloxycarbonyl (Alloc). Suitable carboxyl protecting groups include, but are not limited to, simple esters, such as methyl ester, ethyl ester, tert-butyl ester, and benzyl ester, as well as esters formed with, e.g., trityl, 2,4-dimethoxylbenyl (Dmb), and 9-fluorenylmethyl (Fm). Other suitable protecting groups are known to those of ordinary skill in the art. In some embodiments, amine (A) is selected from phenylalanine, phenylalanine derivatives, substituted phenylalanine, substituted phenylalanine derivatives, tryptophan, tryptophan derivatives, substituted tryptophan, substituted tryptophan derivatives, phenylpropanolamine, protected phenylpropanolamine, substituted phenylpropanolamine, protected substituted phenylpropanolamine, dolaphenine and protected dolaphenine, substituted dolaphenine, protected substituted dolaphenine, dolaphenine derivatives, protected dolaphenine derivatives, substituted dolaphenine derivatives, protected dolaphenine derivatives. In still other embodiments, the amine may be a peptide, preferably having 2-6 amino acids residues, the amino acid residues may include combinations of naturally occurring amino acids, non-standard amino acids, substituted amino acids, and amino acid derivatives, and may include a protecting group.

The term “amino acid” refers to both naturally occurring amino acids, i.e., standard and non-standard amino acids, as well as chemically synthesized amino acids, and includes both L- and D-isomers. Substituted amino acids are amino acids that include one or more substituents, typically on the side chain. Amino acid derivatives are amino acids which the α-amino group or acyl group have been chemically modified. Such modifications may include, for example, the addition of protecting groups, spacers, linkers, or other functional groups that are useful for further modification of the amino acid. Protected amino acids are amino acid derivatives that have a protecting group on the α-amino group, the acyl group or both the α-amino group and the acyl group. Suitable amine protecting groups include, but are not limited to, tert-butoxycarbonyl groups (Boc). 9-fluorenylmethoxycarbonyl groups (Fmoc), benzoyloxycarbonyl groups (Cbz, Z), and Allyloxycarbonyl (Alloc). Suitable carboxyl protecting groups include, but are not limited to, simple esters, such as methyl ester, ethyl ester, tert-butyl ester, and benzyl ester, as well as esters formed with, e.g., trityl, 2,4-dimethoxylbenyl (Dmb), and 9-fluorenylmethyl (Fm).

The term “aryl” refers to a carbocyclic aromatic group. Examples of aryl groups include, but are not limited to, phenyl, naphthyl and anthracenyl. In various embodiments, carboxylic acid (CA) is selected from amino acids, amino acid derivatives, peptides and peptide derivatives.

The term “carboxylic acid (CA)” as used herein refers specifically to the carboxylic-acid containing compound that is reacted with the N-terminal of the universal dolastatin core in the methods described herein. The carboxylic acid (CA) may be selected from amino acids, amino acid derivatives, peptides and peptide derivatives. When carboxylic acid (CA) is a peptide or peptide derivative, it is preferably 2-6 amino acid residues in length. In some embodiments, the carboxylic acid (CA) includes one or more substituents, suitable substituents include C₁-C₆ alkyl, hydroxy, C₁-C₆ alkoxy, amino, thiol, C₁-C₆ alkylthio, and halo. In some embodiments, the carboxylic acid (CA) may have a protecting group. The protecting group may a carboxyl protecting group, be an amine protecting group, or a protecting group on the side chain or other protectable location. Suitable carboxyl protecting groups include, but are not limited to, simple esters, such as methyl ester, ethyl ester, tert-butyl ester, and benzyl ester, as well as esters formed with, e.g., trityl, 2,4-dimethoxylbenyl (Dmb), and 9-fluorenylmethyl (Fm). Suitable amine protecting groups include, but are not limited to, tert-butoxycarbonyl groups (Boc). 9-fluorenylmethoxycarbonyl groups (Fmoc), benzoyloxycarbonyl groups (Cbz, Z), and Allyloxycarbonyl (Alloc). Other suitable protecting groups are known to those of ordinary skill in the art. In some embodiments, carboxylic acid (CA) may include one or more of a spacer, a linker and an attachment group.

The term “coupling agent” refers to peptide coupling reagents used to activate a carboxyl moiety of a carboxylic acid to facilitate reaction with an amino group, such as the α-amino group of an amino acid, or the N-terminal amine of the universal dolastatin core. Suitable coupling agents used in the methods provided herein include, but are not limited to, cabonyldiimidazole (CDI), propylphosphonic anhydride (T3P) solution and HATU. Other suitable coupling agents are known to those of ordinary skill in the art. Coupling agents are generally used in the presence of a base, such as diisopropylethylamine (DIPEA) and N-methylmorpholine (NMM).

The term “coupling additive” refers to peptide coupling reagents that, in addition to facilitating the formation of a peptide bond, also inhibit side reactions and reduce racemization. Useful coupling additives for the reactions described herein include N-hydroxysuccinimide (HOSu), N-hydroxy-5-norbornene-2,3-dicarboximide (HONB), 1-hydroxybenzotriazole (HOBt), 6-chloro-1-hydroxybenzotriazole (6-Cl-HOBO, 1-hydroxy-7-azabenzotriazole (HOAt), 3-hydroxy-4-oxo-3,4-dihydro-1,2,3-benzotriazine (HODhbt), its aza derivative (HODhat), and 2-pyridinol 1-oxide (HOPO). Coupling additives are generally used in the presence of a base, such as 2,6-lutidine, DIPEA and NMM.

The term “halo” refers to elements in VIIa of the periodic table, such as fluorine, chlorine, bromine and iodine. The term “halide” refers to the ion of a halogen.

The term “heterocyclic” refers to any alkyl or aryl ring containing at least one non-carbon atom in the ring, i.e., heteroalkyl and heteroaryl, respectively. Exemplary non-carbon atoms include, but are not limited to, oxygen, nitrogen and sulfur; heterocyclic rings may include two or more non-carbon atoms in the ring; in such instances, the two or more non-carbon atoms may be the same or different.

The term “linker” refers to a chemical entity modified to attach to an antibody or small molecule targeting group at one end and a cytotoxic agent, such as a dolastatin or auristatin at the other end. Conventional linkers include cleavable linkers and non-cleavable linkers. The linkers may include an attachment group, for attachment to an antibody, antibody fragment or other targeting entity, and a spacer, which allows for interaction at, e.g., a cleavable site.

The term “peptide” as used herein, are 2 or more amino acids, substituted amino acids, amino acid derivatives, or substituted amino acid derivatives, including standard, non-standard, and chemically synthesized amino acids, and including for L- and D-isomers, that are linked together in an amide linkage. Peptides may include protecting groups at the N-terminal or the C-terminal. Suitable amine protecting groups include, but are not limited to, tert-butoxycarbonyl groups (Boc), 9-fluorenylmethoxycarbonyl groups (Fmoc), benzoyloxycarbonyl groups (Cbz, Z), and Allyloxycarbonyl (Alloc). Suitable carboxyl protecting groups include, but are not limited to, simple esters, such as methyl ester, ethyl ester, tert-butyl ester, and benzyl ester, as well as esters formed with, e.g., trityl, 2,4-dimethoxylbenyl (Dmb), and 9-fluorenylmethyl (Fm). When peptides, substituted peptides, peptide derivatives, or substituted peptide derivatives are used for amine (A) or carboxylic acid (CA), they are preferably 2-6 amino acid residues in length.

The term “substituent” refers to any group that replaces a hydrogen atom of, e.g., an alkyl, cycloalkyl, aryl, heteroaryl, amine, and so forth. Substituents may include, but are not limited to, such groups as alkyls, substituted alkyls, aryls, heteroaryls, ethers, amines, amides, thiols, sulfides, disulfides, halo and protecting groups. Suitable substituents for the amine (A) and the carboxylic acid (CA) used in the methods described herein include C₁-C₆ alkyl, e.g., methyl, ethyl, propyl, isopropyl, butyl, sec-butyl, isobutyl, tert-butyl, pentyl, and so forth, hydroxy, C₁-C₆ alkoxy, e.g., methoxy, ethoxy, and so forth, amino, thiol, C₁-C₆ alkylthio, and halo.

Examples. The following examples are illustrative of the synthesis of the universal dolastatin core and useful intermediates.

### Example 1. Preparation of N-Boc-Dolaisoleuine Methylbenzylamine Salt

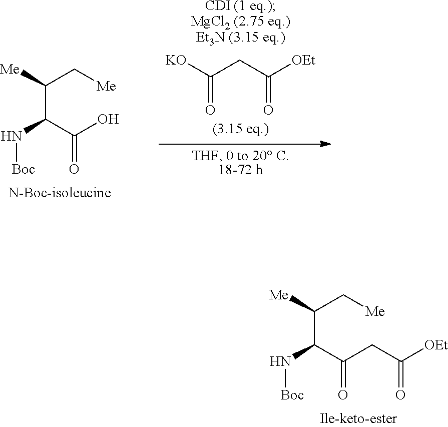

To a cooled solution (7° C.) of N-Boc-isoleucine (100 g, 1 equiv.) in THF (5 vol.) was charged CDI (1 equiv.), portion-wise. The reaction was warmed to 20° C. and stirred for 4 h, then cooled to 0° C. To a suspension of potassium mono-ethyl malonate (2.2 equiv.) in THF (15 vol.) at 7° C. was charged anhydrous MgCl₂ (2.75 equiv.), portion-wise, then warmed to 20° C. and stirred for 1 h. This suspension was cooled to 0° C., charged with Et₃N (3.15 equiv.), and stirred for 2 h at 0° C. The 0° C. imidazolide solution was charged slowly to the malonate suspension maintaining the temperature ≤3° C. The combined suspension was warmed to 20° C. and stirred for 18-72 h. The reaction was quenched with 10% (w/w) aqueous citric acid (20 vol.), maintaining an internal temperature of ≤23° C., and then concentrated under reduced pressure. The acidic aqueous layer was extracted with MTBE (3×5 vol.). The combined organic extracts were washed with 20% (w/w) aqueous Na₂CO₃ (2×5 vol.), dried over Na₂SO₄ and concentrated under reduced pressure to give the desired Ile-keto-ester (95% yield) without further purification.

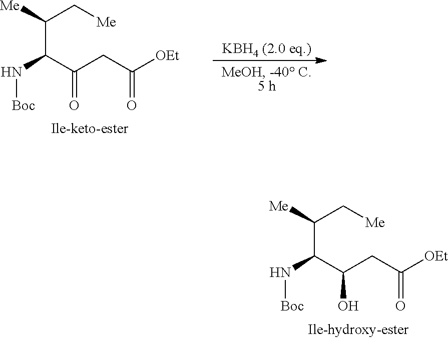 [SMILES: CCOC(=O)CC(=O)[C@@H](NC(=O)OC(C)(C)C)[C@@H](C)CC ; "KBH4 (2.0 eq.)" ; "MeOH, -40° C.5 h" ; CCOC(=O)C[C@@H](O)[C@@H](NC(=O)OC(C)(C)C)[C@@H](C)CC]

MeOH (5 vol.) was cooled to ≤−40° C. and KBH₄ (2.0 equiv.) was charged and stirred at −40° C. for 30 min. A solution of Ile-keto-ester (130 g, 1 equiv.) in MeOH (5 vol.) and charged slowly to the KBH₄ slurry, maintaining the internal temperature ≤−40° C. The reaction was stirred for 5 h at −40° C. The reaction was quenched by charging to a stirred solution of 10% (w/w) aqueous citric acid (20 vol.), with internal temperature ≤8° C. and a resulting pH of 3-5. The MeOH was removed under reduced pressure, and the remaining aqueous phase extracted with MTBE (3×5 vol.). The combined organic extracts were washed with 20% (w/w) aqueous Na₂CO₃ (2×5 vol.), dried over Na₂SO₄, and concentrated under reduced pressure to give the desired Ile-hydroxy-ester (89% yield, dr≥13:1) without further purification.

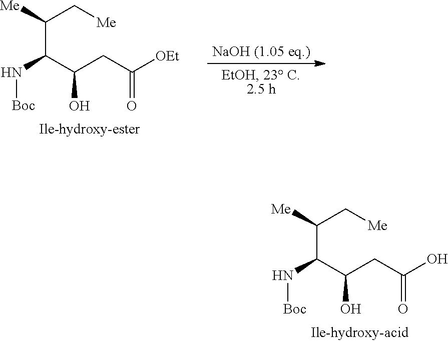 [SMILES: CCOC(=O)C[C@@H](O)[C@@H](NC(=O)OC(C)(C)C)[C@@H](C)CC ; "EtOH, 23° C.2.5 h" ; "NaOH (1.05 eq.)" ; CC[C@H](C)[C@H](NC(=O)OC(C)(C)C)[C@H](O)CC(=O)O]

To a RT solution of Ile-hydroxy-ester (118 g, 1 equiv.) in EtOH (5 vol.) was charged 2.5 M aqueous NaOH (1.05 equiv.). The reaction was stirred with an internal temperature ≤23° C. for 2.5 h. EtOH was removed under reduced pressure and the basic aqueous phase extracted with MTBE (2×5 vol.). The combined organic phases were extracted with 2.5 M aqueous NaOH (2×0.5 vol.). The aqueous phases were combined and acidified with H₃PO₄ (85 wt %, 1.5 equiv.) to a pH of 4. The acidified aqueous phase was extracted with MTBE (3×5 vol.). The combined organic extractions were dried over Na₂SO₄ and concentrated under reduced pressure to a constant weight of crude Ile-hydroxy-acid (99% yield). The resultant weight of the crude was used in the subsequent crystallization.

The crude Ile-hydroxy-acid was dissolved in MTBE (2 vol.) and heptane (2 vol.), and then warmed to 55° C. Heptane (4 vol.) was charged, maintaining the temperature at 55° C. The mixture was cooled to 45° C. and seeded (0.5 wt %). After initiation of crystallization was observed, the mixture was held at 45° C. for 2 h, cooled to RT over 1 h, stirred vigorously for another 12 h, and then the solid products were isolated by filtration. The filter cake was washed with heptane (2 vol.) and dried under vacuum to give the desired Ile-hydroxy-acid (75% yield, dr>99:1). The crystallization step eliminates the need for purification by column chromatography, providing both a stable, solid product and scalability.

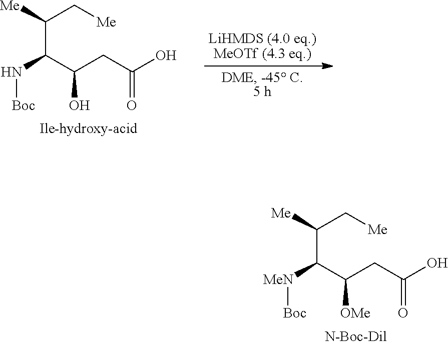 [SMILES: CC[C@H](C)[C@H](NC(=O)OC(C)(C)C)[C@H](O)CC(=O)O ; "DME, -45° C.5 h" ; "LiHMDS (4.0 eq.)MeOTf (4.3 eq.)" ; CC[C@H](C)[C@@H]([C@@H](CC(=O)O)OC)N(C)C(=O)OC(C)(C)C]

To a chilled (−45° C.) solution of Ile-hydroxy-acid (5 g, 1 equiv.) in anhydrous dimethoxyethane (20 vol.) was charged LiHMDS (1.0 M in hexanes, 4.0 equiv.), maintaining the internal temperature ≤−45° C. MeOTf (4.3 equiv.) was then charged, maintaining the internal temperature at ≤−45° C. The reaction was stirred at ≤−45° C. for 5 h until judged complete. The reaction was quenched by the addition of methanol (5 vol.) and 10% (w/w) aqueous NaOH (4.0 equiv.), maintaining an internal temperature of ≤−45° C., then warmed to 5° C. and stirred until judged complete. The reaction mixture was concentrated under reduced pressure to remove organic solvents and then diluted with heptane (20 vol.), which was extracted with 10% (w/w) aqueous NaOH (2×5 vol.) The combined basic aqueous phases were adjusted to pH 4 by the addition of H₃PO₄ and then extracted with 1:1 heptane/MTBE (3×5 vol.). The combined organic layers were dried over Na₂SO₄ and concentrated under reduced pressure to crude N-Boc-Dil. The resultant weight of the isolated crude was used in the subsequent chromatographic step.

The crude N-Boc-Dil was loaded onto silica (not less than 20 g silica per 1 g of crude; silica equilibrated with eluent) using a minimal amount of 1:5 MTBE/heptane and eluted with 20% MTBE/heptane/0.1% acetic acid. Positive fractions were combined and reduced under pressure, and the resultant weight was used for the subsequent recrystallization.

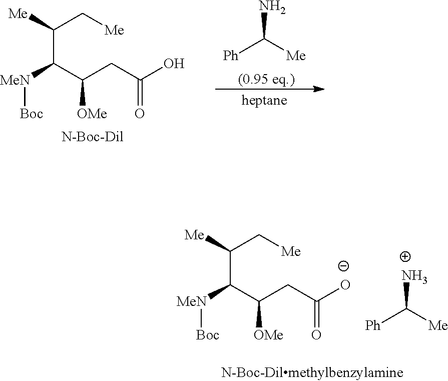

N-Boc-Dil (1 equiv.) was dissolved in heptane (10 vol.) and heated to 50° C. for 30 min. S-α-methylbenzylamine (0.95 equiv.) was charged, the reaction was cooled to 37° C., and then seeded (0.5 wt %). The reaction was cooled to 20° C. over 1 h and stirred for an additional 6 h, and then solid products were isolated by filtration. The filter cake was washed with heptane (2 vol.) and dried under vacuum to give the final N-Boc-Dil.methylbenzylamine.

### Example 2. Preparation of O-benzyl Ester Dolaproine Hydrochloride Salt

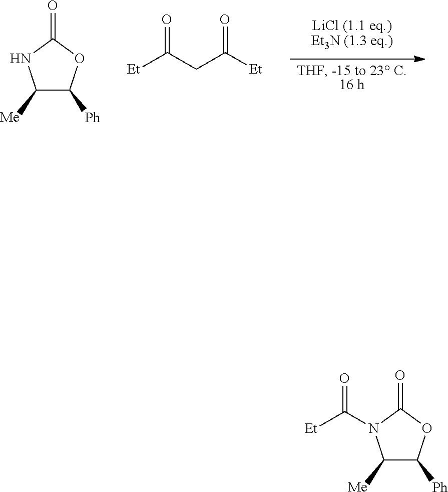 [SMILES: C[C@H]1NC(=O)O[C@H]1c1ccccc1 ; CCC(=O)CC(=O)CC ; "LiCl (1.1 eq.)Et3N (1.3 eq.)" ; "THF, -15 to 23° C.16 h" ; CCC(=O)N1C(=O)O[C@@H](c2ccccc2)[C@H]1C]

A solution of (4R,5S)-(+)-4-methyl-5-phenyl-2-oxazolidinone (100.0 g, 1 equiv.) in THF (30 vol.) was cooled to −20° C. To the precooled solution was added lithium chloride (1.1 equiv.) followed by Et₃N (1.3 equiv.) while maintaining an internal temperature <−15° C. Propionic anhydride (1.2 equiv.) was added over 30 min. while maintaining an internal temperature <−15° C. The mixture was warmed to 23° C. and stirred for 16 h. The reaction was concentrated under reduced pressure and partitioned between EtOAc (5 vol.) and 0.2 M aqueous HCl (5 vol.). The organic layer was washed with 1 M aqueous NaHCO₃ (2×2 vol.) and brine (2×1 vol.). The organic layer was dried over Na₂SO₄, filtered and concentrated under reduced pressure to afford the desired Evans-type oxazolidinone (130.1 g, 99%) as a viscous, light yellow oil which was used without further purification.

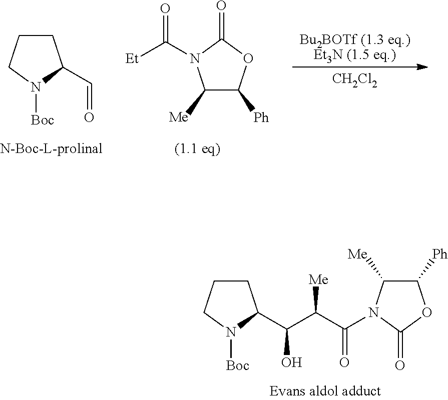 [SMILES: CC(C)(C)OC(=O)N1CCC[C@H]1C=O ; CCC(=O)N1C(=O)O[C@@H](c2ccccc2)[C@H]1C ; "CH2Cl2" ; "Bu2BOTf (1.3 eq.)Et3N (1.5 eq.)" ; C[C@@H]1[C@H](c2ccccc2)OC(=O)N1C(=O)[C@H](C)[C@@H](O)[C@@H]1CCCN1C(=O)OC(C)(C)C]

A solution of (4R,5S)-4-methyl-5-phenyl-3-propionyloxazolidin-2-one (25.75 g, 1.1 equiv.) in CH₂Cl₂ (10 vol.) was cooled to 0° C. Triethylamine (1.5 equiv.) was charged to this cold reaction mixture followed by the addition of dibutyl boron triflate (1 M in CH₂Cl₂, 1.3 equiv.) while maintaining the reaction temperature <4° C. The reaction was stirred for 1 h at 0° C. then cooled to −70° C. A solution of N-Boc-L-prolinal (20.0 g, 1.0 equiv.) in CH₂Cl₂ (6 vol.) was charged, maintaining the temperature <−60° C. The reaction was stirred for 2 h at −70° C., 1 h at −0° C., then 15 min at room temperature. The reaction was quenched with 0.1 M aqueous sodium phosphate buffer (pH=7, 8 vol.) followed by the slow addition of 30% aqueous H₂O₂/MeOH (1:2, 30 vol.) maintaining the temperature <10° C. and stirred for 1 h. The mixture was diluted with DI water (15 vol.) and concentrated under reduced pressure to complete removal of organic solvent. DI water (15 vol.) was added to the residue. The mixture was extracted with EtOAc (3×15 vol.). The combined organics were washed with 1 M KHSO₄ (15 vol.), DI water (15 vol.), saturated aqueous NaHCO₃ (15 vol.) and brine (15 vol.). Charcoal (20 wt %) was charged and removed by filtration. The filtrate was concentrated under reduced pressure to obtain Evans aldol adduct as a white foam that was utilized without further purification (45 g).

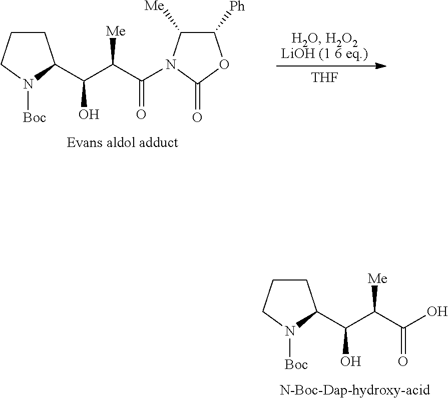 [SMILES: C[C@@H]1[C@H](c2ccccc2)OC(=O)N1C(=O)[C@H](C)[C@@H](O)[C@@H]1CCCN1C(=O)OC(C)(C)C ; "H2O, H2O2LiOH (1 6 eq.)" ; C[C@@H](C(=O)O)[C@@H](O)[C@@H]1CCCN1C(=O)OC(C)(C)C ; "THF"]

To a solution of Evans aldol adduct (50 g, 1.0 equiv.) in anhydrous THF (13 vol.) was charged water (3.3 vol.) and cooled to 5° C. 30% aqueous H₂O₂ (1 vol.) was added, followed by the addition of LiOH (1.6 equiv.) in DI water (2 vol.). The reaction mass was stirred for 5 h at 5° C. under nitrogen atmosphere. The reaction was quenched by the addition of NaHSO₃ (4 equiv.) in DI water (5 vol.) and stirred for 16 h. The resulting mixture was adjusted to pH 9 with saturated aqueous NaHCO₃ and washed with CH₂Cl₂ (2×10 vol.). The aqueous layer was cooled and adjusted to pH 2 by addition of 1 M aqueous KHSO₄ and extracted with EtOAc (2×10 vol.). The combined EtOAc extracts were washed with brine (10 vol.) then dried over Na₂SO₄ and concentrated under reduced pressure to obtain N-Boc-Dap-hydroxy-acid as a yellow oil that was used without further purification (24 g).

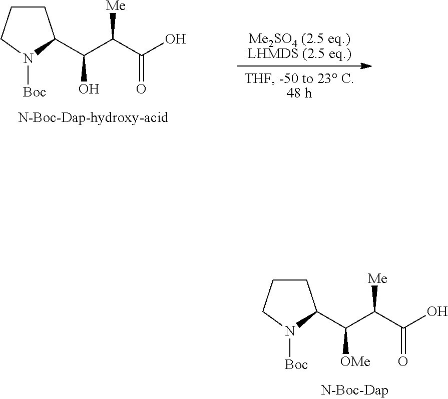 [SMILES: C[C@@H](C(=O)O)[C@@H](O)[C@@H]1CCCN1C(=O)OC(C)(C)C ; "Me2SO4 (2.5 eq.)LHMDS (2.5 eq.)" ; "THF, -50 to 23° C.48 h" ; CO[C@H]([C@@H](C)C(=O)O)[C@@H]1CCCN1C(=O)OC(C)(C)C]

To a −50° C. solution of N-Boc-Dap-hydroxy-acid (6.0 g, 1.0 equiv.) in anhydrous THF (20 vol.) was added Me₂SO₄ (2.5 equiv.) followed by LiHMDS (1 M in THF, 2.5 equiv.) maintaining the temperature <−50° C. The reaction was warmed to room temperature and stirred for 48 h under nitrogen atmosphere. The reaction was quenched by the addition of 10% aqueous NaOH and stirred for 12 h. The mixture was concentrated under reduced pressure to remove THF and adjusted to pH 4 by the addition of 1 M aqueous H₃PO₄. The acidified aqueous layer was extracted with MTBE (3×10 vol.) and concentrated under reduced pressure to obtain N-Boc-Dap as a yellow oil which was used without further purification (5.5 g).

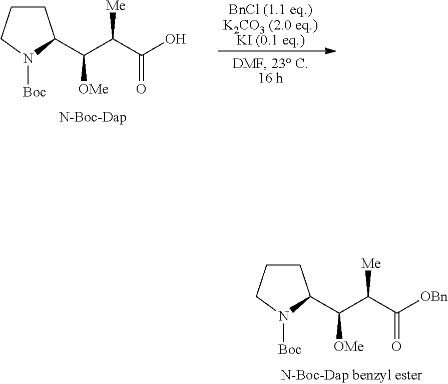 [SMILES: CO[C@H]([C@@H](C)C(=O)O)[C@@H]1CCCN1C(=O)OC(C)(C)C ; "BnCl (1.1 eq.)K2CO3 (2.0 eq.)KI (0.1 eq.)" ; "DMF, 23° C.16 h" ; CO[C@H]([C@@H](C)C(=O)OCc1ccccc1)[C@@H]1CCCN1C(=O)OC(C)(C)C]

To a solution of N-Boc-Dap (2.45 g, 1 equiv.) in DMF (8 vol.) was added K₂CO₃ (2.0 equiv.), KI (0.1 equiv.) and benzyl chloride (1.1 equiv.). The reaction was stirred for 16 h at RT and then quenched by the addition of toluene (5 vol.) and DI water (5 vol.). The organic layer was collected and the aqueous layer extracted with toluene (5 vol.). The combined organic layers were washed with DI water (5 vol.) and concentrated under reduced pressure. The residue was purified via silica gel column chromatography (Sfär 50 g duo) eluting with a heptane to 60:40 heptane/EtOAc gradient to obtain N-Boc-Dap benzyl ester as a colorless oil (2.84 g, 88%).

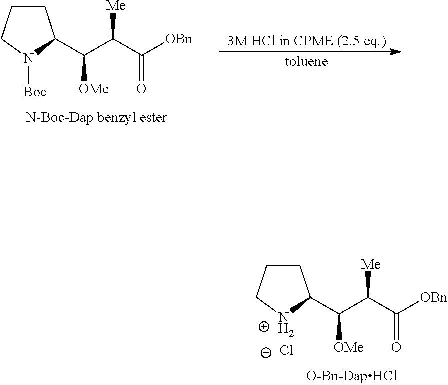 [SMILES: CO[C@H]([C@@H](C)C(=O)OCc1ccccc1)[C@@H]1CCCN1C(=O)OC(C)(C)C ; "3M HCl in CPME (2.5 eq.)" ; "toluene" ; CO[C@@H]([C@@H]1CCC[NH2+]1)[C@@H](C)C(=O)OCc1ccccc1 ; [Cl-]]

To a stirred solution of N-Boc-Dap benzyl ester (5.7 g, 1 equiv.) in toluene (5 vol.) was added 3 M HCl in CPME (2.5 equiv.). After stirring for 16 h, the reaction was concentrated under reduced pressure to removed CPME and HCl. The residue was dissolved in toluene (5 vol.) and heated to 80° C. After dissolution, the reaction was cooled to RT and the solid product isolated by filtration. The solid was washed with toluene (2 vol.) and dried to afford O-Bn-Dap.HCl (2.9 g, 61%) as a white crystalline solid.

### Example 3. Preparation of Dil-Dap-O-Bn.HCl

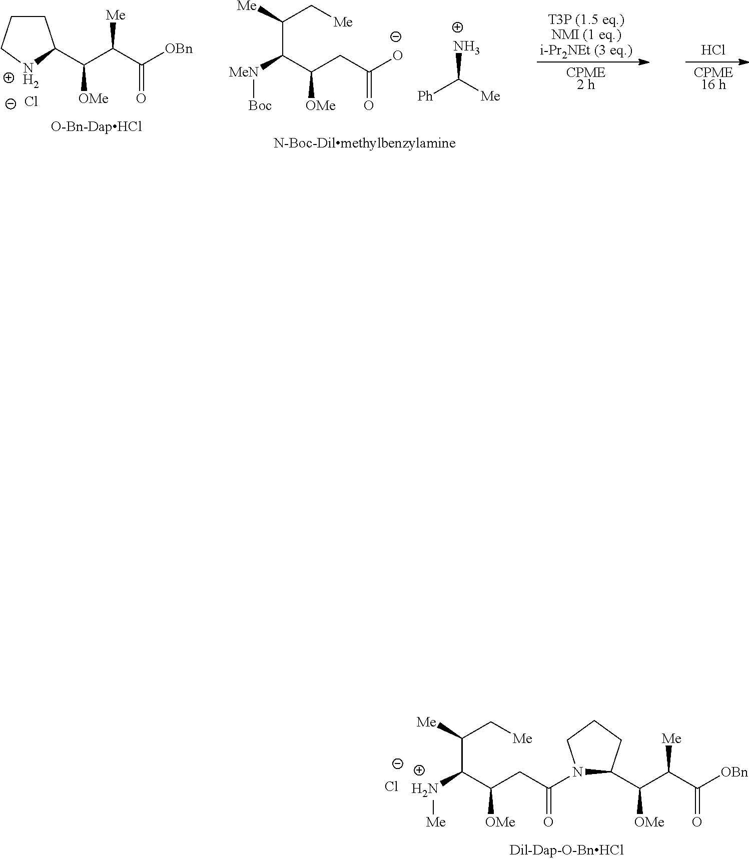

A stirred mixture of N-Boc-Dil.methylbenzylamine (10 g, 1 equiv.) in CPME (4 vol.) was washed with 4 M aqueous HCl (3×1.5 vol.) and DI water (3 vol.). The combined aqueous washes were extracted with CPME (3 vol.) and the combined organic layers dried by constant volume distillation (10 vol.). To the stirred solution was added OBn-Dap.HCl (1 equiv.), NMI (1 equiv.), T3P (50% solution in EtOAc, 1.5 equiv.) and DIPEA (3 equiv.). After stirring for 2 h the reaction was quenched by the addition of 4 M aqueous HCl (3 vol.) and the layers separated. The organic layer was washed with 4 M aqueous HCl (3 vol.) then DI water (3 vol.). The combined aqueous layers were extracted with CPME (3 vol.) then the combined organic layers dried by constant volume distillation (10 vol.). To the stirred solution was added 3 M HCl in CPME (5 equiv.). After stirring for 16 h the reaction was concentrated. The residue was crystallized from MTBE/CPME to give Dil-Dap-OBn.HCl as a white solid (8.6 g, 68%, 99.6 A%).

### Example 4. Preparation of Isolated Universal Dolastatin Core N-Boc-Val-Dil-Dap-OH.DCHA from Dil-Dap-OBn.HCl

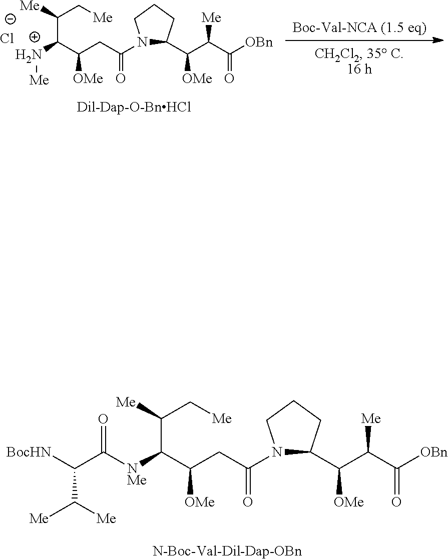 [SMILES: [Cl-] ; CC[C@H](C)[C@H]([NH2+]C)[C@@H](CC(=O)N1CCC[C@H]1[C@H](OC)[C@@H](C)C(=O)OCc1ccccc1)OC ; "Boc-Val-NCA (1.5 eq)" ; "CH2Cl2, 35° C.16 h" ; CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)OC(C)(C)C)C(C)C]

A solution of Dil-Dap-OBn.HCl in CH₂Cl₂ (4 vol.) was washed with 20% aqueous Na₂CO₃ (3×2 vol.) and the aqueous layer extracted with CH₂Cl₂ (2 vol.). The combined organic layers were washed with DI water (3 vol.) and dried by constant volume distillation (10 vol.). N-Boc-Val-NCA (1.5 equiv.) was added to the stirred solution and the reaction stirred at 35° C. for 16 h. The solvent was swapped to iPrOAc and the reaction quenched by the addition of 1 M aqueous NaHCO₃ (5 vol.) and glycine (5 equiv.). The organic layer was washed with 1 M NaHCO₃ (5 vol.) and DI water (5 vol.). The N-Boc-Val-Dil-Dap-OBn product was utilized in the subsequent step as a solution in iPrOAc.

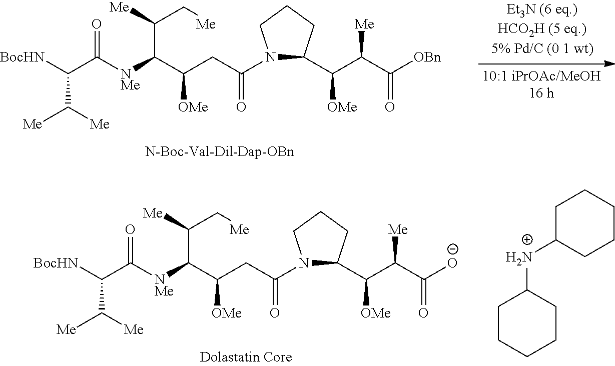

To a stirred solution N-Boc-Val-Dil-Dap-OBn (2 g, 1 equiv.) in iPrOAc (10 vol.) was added MeOH (1 vol.), 5% Pd/C (0.1 wt.), TEA (6 equiv.) and formic acid (5 equiv.). After 16 h the solution was filtered through Celite and filtrate washed with 4 M aqueous HCl (3×5 vol.) and DI water (5 vol.). The solvent was swapped to 80:20 heptane/iPrOAc and DCHA (1 equiv.) was charged. Dolastatin Core, N-Boc-Val-Dil-Dap-OH.DCHA, (75%, 99 A %) was isolated as a white crystalline solid by filtration.

### Example 5. Full Preparation of the Universal Dolastatin Core

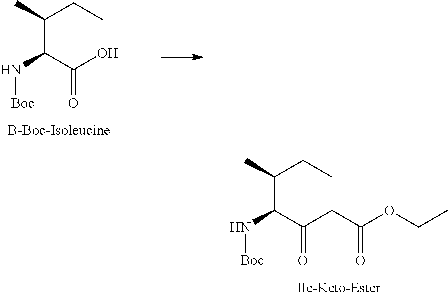 [SMILES: CC[C@H](C)[C@H](NC(=O)OC(C)(C)C)C(=O)O ; CCOC(=O)CC(=O)[C@@H](NC(=O)OC(C)(C)C)[C@@H](C)CC]

To a cooled solution (0° C.) of N-Boc-isoleucine (1 eq.) in THF (5 vol.) was charged CDI (1 eq.) portion wise. The reaction was allowed to warm to RT and stirred for 3 h. To a suspension of potassium monoethyl malonate (2.2 eq.) in THF (15 vol.) at 0° C. was added Et₃N (3.15 eq.) and anhydrous MgCl₂ (2.75 eq.). The suspension was allowed to warm to RT, stirred for 3 h then cooled to 0° C. The RT imidazolide solution was charged slowly to the malonate suspension maintaining the temperature at NMT 5° C. The combined suspension was allowed to warm to RT and stirred for 72 h. The reaction was quenched with 10% aqueous citric acid (20 vol.) and concentrated under reduced pressure to remove THF. The acidic aqueous layer was extracted with MTBE (3×5 vol.). The combined organic extracts were washed with sat. aq. NaHCO₃ (5 vol.), dried over Na₂SO₄ and concentrated under reduced pressure to give the desired product (95% yield) without further purification.

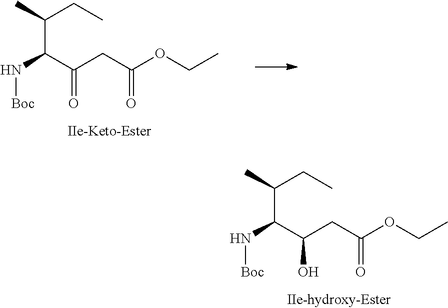 [SMILES: CCOC(=O)CC(=O)[C@@H](NC(=O)OC(C)(C)C)[C@@H](C)CC ; CCOC(=O)C[C@@H](O)[C@@H](NC(=O)OC(C)(C)C)[C@@H](C)CC]

Methanol (5 vol.) was cooled to NMT −40° C. and KBH₄ (2 eq.) was charged and stirred at NMT −40° C. for 30 min. Keto-Ester (1 eq.) was dissolved in Methanol (5 vol.) and charged slowly to the KBH₄ slurry maintaining the internal temperature NMT −40° C. The reaction was stirred for 4 h at that temperature then quenched by charging to a stirred solution of 10% aqueous citric acid (20 vol.) The pH of the resulting solution was 3-5. The MeOH was removed under reduced pressure then the aqueous layer extracted with MTBE (3×5 vol.). The combined organic extracts were washed with sat. aq. NaHCO₃ (5 vol.), dried over Na₂SO₄ and concentrated under reduced pressure to give the desired product (89% yield) without further purification (diastereoselectivity 13:1).

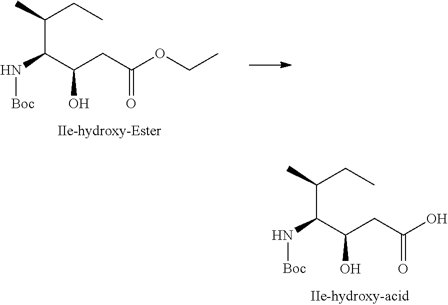 [SMILES: CCOC(=O)C[C@@H](O)[C@@H](NC(=O)OC(C)(C)C)[C@@H](C)CC ; CC[C@H](C)[C@H](NC(=O)OC(C)(C)C)[C@H](O)CC(=O)O]

To a RT solution of Ile-hydroxy-ester (1 eq.) in EtOH (5 vol.) was charged 10% aqueous NaOH (1.05 eq.) and diluted with water (4 vol.). The reaction was stirred at RT for 2.5 h after which time HPLC indicated the reaction was complete. The EtOH was removed under reduced pressure and the basic aqueous phase extracted with MTBE (2×5 vol.). The combined organic phases were extracted with 10% aqueous NaOH (0.5 vol.) and the aqueous extract combined with the product containing basic aqueous phase. The combined aqueous phase was acidified with H₃PO₄ (85 wt %, 1.5 eq.) to adjust the pH to 4. The acidified aqueous phase was extracted with MTBE (3×5 vol.). The combined organic extracts were dried over Na₂SO₄ and concentrated under reduced pressure to a constant weight (99% yield).

The weight of the isolated product was used in the subsequent crystallization. The crude Ile-hydroxy-acid was dissolved in MTBE (2 vol.) and Heptane (2 vol.) and the solution warmed to NMT 55° C. Heptane (4 vol.) was charged maintaining the temperature NLT 50° C. After the addition the mixture was cooled to 45° C. Upon cooling to 45° C. the crystallization was initiated, (either spontaneous, or addition of 0.5 wt % of seed). After crystallization initiation was observed the mixture was held at 45° C. for 2 h then cooled to RT. After cooling to RT the slurry was stirred vigorously for 12 h then isolated by filtration. The filter cake was washed with heptane (2 vol.) and dried under vacuum. The product was isolated in 75% yield with a dr >99:1.

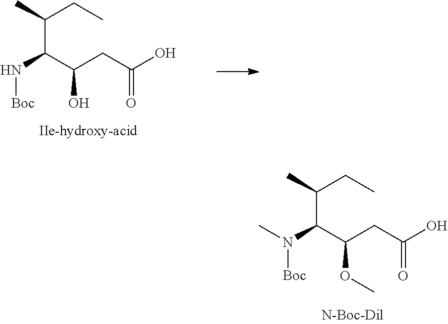 [SMILES: CC[C@H](C)[C@H](NC(=O)OC(C)(C)C)[C@H](O)CC(=O)O ; CC[C@H](C)[C@@H]([C@@H](CC(=O)O)OC)N(C)C(=O)OC(C)(C)C]

A solution of Ile-hydroxy-acid (1 eq.) in anhydrous THF (20 vol.) was cooled to NMT −50° C. and Me₂SO₄ (2.05 eq.) was charged maintaining the temperature NMT −50° C. LiHMDS (1.0 M in THF, 3.3 eq.) was charged maintaining the temperature NMT −50° C. The reaction was allowed to warm to RT and stirred overnight. The reaction was quenched by the addition of 10% aqueous NaOH (10 eq.) and stirred for 12 h. The mixture was concentrated under reduced pressure to remove THF then the basic aqueous phase was extracted with MTBE (2×5 vol.). The aqueous phase was adjusted to pH 4 by the addition of H₃PO₄ and extracted with MTBE (3×5 vol.). The combined organic layers were dried over Na₂SO₄ and concentrated under reduced pressure, the crude material was isolated in good yield. N-Boc-Dil was further purified by crystallization in the subsequent step.

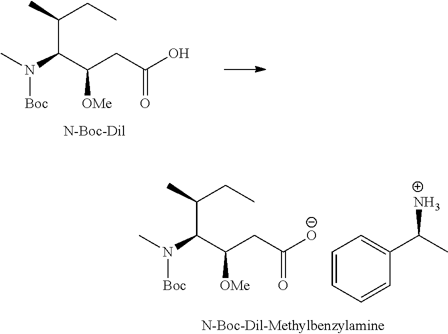

N-Boc-Dil (1 eq.) was dissolved in heptane (10 vol.) and heated to 50° C. S-α-methylbenzylamine (1 eq.) was charged and the reaction cooled to 35° C. After stirring at 35° C. for 1 h the reaction was cooled to RT and stirred vigorously for 12 h. The solid was isolated by filtration and washed with heptane (2 vol.) to give the salt in high yield (70%).

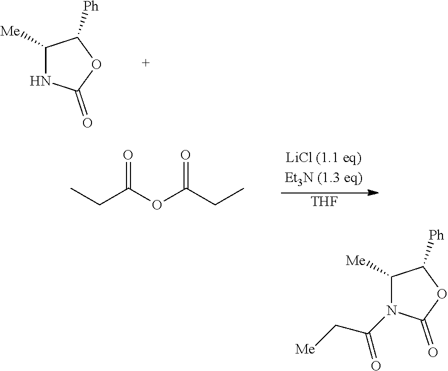

(4R,5S)-4-methyl-5-phenyl-3-propionyloxazolidine. A solution of (4R,5S)-(+)-4-methyl-5-phenyl-2-oxazolidinone (100.0 g, 0.564 mol, 1 equiv) in 30 vol. of tetrahydrofuran was cooled to −20° C. in a 70% water-MeOH/dry ice bath. To the precooled solution was added lithium chloride (26.32 g, 0.621 mol, 1.1 equiv) followed by triethylamine (102.2 mL, 0.734 mol, 1.3 equiv) at such a rate to keep the temperature below −15° C. Propionic anhydride (86.82 mL, 0.677 mol, 1.2 equiv) was then added over 30 min to keep the temperature below −15° C. The mixture was then removed from the bath and stirred at room temperature overnight (˜15 h). Once the reaction was complete (confirmed by LCMS or HPLC), the mixture was concentrated under reduced pressure. The mixture was then partitioned between 500 mL of ethyl acetate and 500 mL of 0.2 M hydrochloric acid. The organic layer was then collected and washed with 2×100 mL of 1 M sodium bicarbonate and 2×100 mL brine. The organic layer was collected and dried over sodium sulfate. Concentration under reduced pressure afforded the final product (130.1 g, 0.558 mol, 99%) as a viscous, slightly yellow oil. No purification was required.

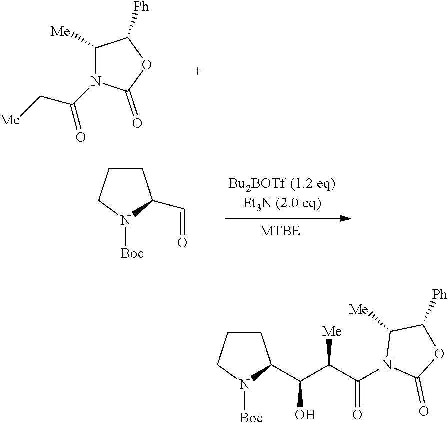

N-Boc-Pro-Xc. A solution of (4R,5S)-4-methyl-5-phenyl-3-propionyloxazolidine (35.9 g, 0.164 mol, 1.3 equiv) in 4 vol of MTBE was cooled to 0° C. in an ice bath. The cooled solution was charged with triethylamine (34.8 mL, 0.201 mol, 2.0 equiv) followed by careful addition of 1.0 M dibutylboryl trifluoromethanesulfonate solution in dichloromethane (150.0 mL, 0.150 mol, 1.20 equiv) at such a rate to keep the temperature below 5° C. The mixture was allowed to stir for 5 hrs. After 5 hrs, the solution was cooled to −78° C. in an acetone/dry ice bath. Next a solution of Boc-L-prolinal (25.0 g, 0.125 mol, 1.0 equiv) in 25 mL of MTBE was prepared and cooled to −78° C. The cooled solution was then cannulated to the reaction mixture at such a rate to keep the temperature below −60° C. The reaction was allowed to slowly warm to room temperature for 24 hrs. The reaction was then quenched with phosphate buffer (pH=7, 50 mL), followed by the addition of 75 mL of methanol, and then cooled to 0° C. To the cooled solution was added a solution of 30% aqueous hydrogen peroxide and methanol (1:2, 100 mL) at such a rate to keep the internal temperature below 10° C. The reaction was then stirred at 0° C. for 1 hr. The reaction was quenched with 100 mL of water, then concentrated under reduced pressure. The aqueous mixture was then extracted with MTBE (2×100 mL) and the organic layers are combined. The organic layer was then washed with 1 M potassium bisulfate (2×30 mL), saturated sodium bicarbonate (2×30 mL), and brine (2×30 mL). The organic layer was then collected and dried over sodium sulfate then concentrated under reduced pressure. The crude product was then purified via Si column chromatography using 10% to 30% ethyl acetate in heptane to afford the syn-adduct (11.62 g, 0.027 mol, 22%) as a white foam.

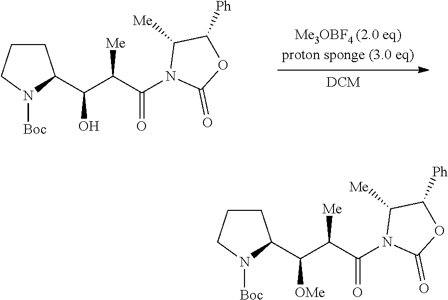 [SMILES: C[C@@H]1[C@H](c2ccccc2)OC(=O)N1C(=O)[C@H](C)[C@@H](O)[C@@H]1CCCN1C(=O)OC(C)(C)C ; CO[C@H]([C@@H](C)C(=O)N1C(=O)O[C@@H](c2ccccc2)[C@H]1C)[C@@H]1CCCN1C(=O)OC(C)(C)C]

N-Boc-Dap-Xc. A solution of N-Boc-Dap-Xc (19.0 g, 0.044 mol, 1.0 equiv) in 5 vol of dichloromethane was cooled to 0° C. in an ice bath. To the cooled solution was added proton-sponge (28.3 g, 0.132 mol, 3.0 equiv) followed by trimethyloxonium tetrafluoroborate (12.85 g, 0.087 mol, 2.0 eq). The reaction mixture was allowed to warm to room temperature overnight (˜18 h). The reaction was cooled to 0° C. and quenched by the addition of 30 mL of water that has been cooled to 0° C. The reaction was then filtered through a pad of celite, and the filtrate was evaporated under reduced pressure and partitioned between 250 mL of MTBE and 200 mL of water. The organic layer was separated, and the aqueous layer was extracted with 3×20 mL of MTBE. The organic layers were combined and washed with 1 M aqueous potassium bisulfate solution (2×20 mL), saturated sodium bicarbonate solution (2×20 mL), and brine (2×20 mL). The organic layer was then dried over sodium sulfate and concentrated under reduced pressure to afford the crude product as an oil. The product was then purified via Si column chromatography (0% to 15% ethyl acetate in heptane). N-Boc-Dap-Xc (11.54 g, 0.026 mol, 59%) was isolated as a clear oil.

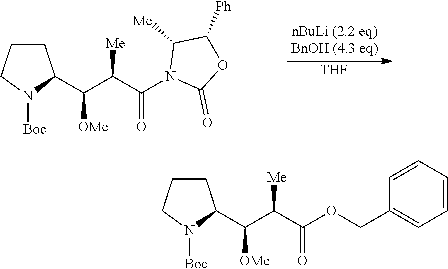 [SMILES: CO[C@H]([C@@H](C)C(=O)N1C(=O)O[C@@H](c2ccccc2)[C@H]1C)[C@@H]1CCCN1C(=O)OC(C)(C)C ; CO[C@H]([C@@H](C)C(=O)OCc1ccccc1)[C@@H]1CCCN1C(=O)OC(C)(C)C ; "THF"]

N-Boc-Dap-OBn. A solution of n-BuLi (2.5M in hexanes, 4.93 mL, 0.012 mol, 2.2 equiv) in THF (9.2 mL) was cooled to 0° C. Benzyl alcohol (2.55 mL, 0.025 mol, 4.3 eq) was added dropwise to the solution. The reaction was allowed to stir at 0° C. for 1 hr. Meanwhile, a 0.2 M solution of N-Boc-Dap-Xc (2.56 g, 5.73 mmol, 1.0 equiv) in THF was prepared and cooled to 0° C. The BnOLi-BnOH was cannulated into the N-Boc-Dap-Xc solution at such a rate to maintain the reaction temperature below 5° C. The reaction was allowed to stir for 1-3 h, until complete by HPLC. The reaction mixture was diluted with 15 mL of ethyl acetate and quenched via the slow addition of 15 mL of sodium bicarbonate. The organic layer was separated and washed with an additional 15 mL of sodium bicarbonate followed by 2×15 mL of water and 2×15 mL of brine. The organic layer was then collected and dried over sodium sulfate. The solution was concentrated under rotary evaporation to afford the crude product. The crude product was purified via Si column chromatography using a 0-50% EtOAc/Heptane gradient. The product (1.78 g, 4.72 mmol, 82%) was isolated as a clear oil.

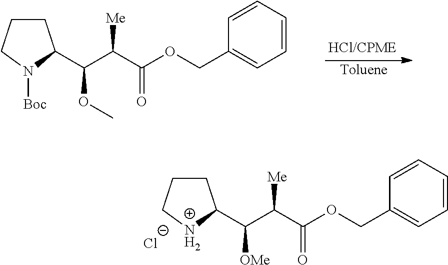 [SMILES: CO[C@H]([C@@H](C)C(=O)OCc1ccccc1)[C@@H]1CCCN1C(=O)OC(C)(C)C ; "Toluene" ; [Cl-] ; CO[C@@H]([C@@H]1CCC[NH2+]1)[C@@H](C)C(=O)OCc1ccccc1]

OBn-Dap-HCl Salt. N-Boc-Dap-OBn (5.7 g, 15 mmol, 1.0 equiv) was dissolved in 5 vol of toluene. To the solution was added 3 M hydrogen chloride solution in CPME (12.6 mL, 2.5 equiv). The reaction mixture was allowed to stir overnight. Upon completion, the solution was concentrated under rotary evaporation. The crude material was taken up in 5 vol of toluene, and heated to 80° C. then allowed to cool to RT. After stirring for 12 h at RT the product was isolated by filtration. The filter cake was washed with 2 vol. of toluene to give the product as a white solid (2.9 g, >99% purity, 62%).

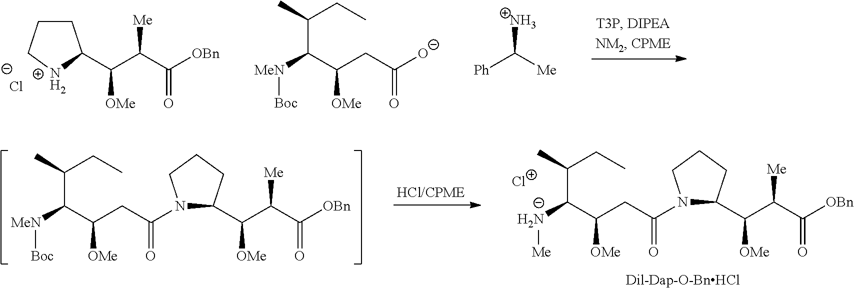

N-Boc-Dil.amine salt (1 eq.) was dissolved in CPME (10 vol.) and washed three times with 2.0 M HCl (5 vol.) and once with H₂O, the combined aqueous washes were extracted with CPME (10 vol.) and the combined organic layers concentrated to 10 vol then constant volume vacuum distilled until water content was <0.1%. OBn-Dap.HCl (1 eq.) was charged followed by DIPEA (3.0 eq), 1-methylimidazole (1.0 eq), and T3P (1.5 eq.). The reaction was stirred at RT O/N at which time HPLC analysis indicated the desired intermediate was formed. The reaction was washed twice with 2.0 M HCl (5 vol.) and twice with water (5 vol.) then constant volume vacuum distilled until water content was <0.1%. 3.0 M HCl in CPME (7.5 eq.) was charged and the reaction stirred at RT O/N at which time HPLC analysis indicated the desired product was formed. Constant volume vacuum distillation to remove HCl and swap the solvent to MTBE was performed and the product recrystallized from MTBE. The product was isolated by filtration as a white solid (70% yield).

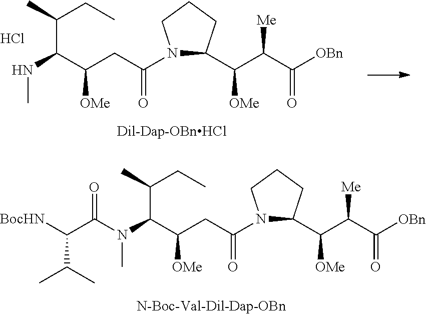 [SMILES: Cl ; CC[C@H](C)[C@H](NC)[C@@H](CC(=O)N1CCC[C@H]1[C@H](OC)[C@@H](C)C(=O)OCc1ccccc1)OC ; CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)OC(C)(C)C)C(C)C]

Dil-Dap-OBn.HCl was dissolved in DMF (15 vol.). To this solution, was charged N-Boc-Val (1.5 eq.), DIPEA (3 eq.) and COMU (1.9 eq.). The reaction was stirred at RT O/N. After this time the reaction was quenched by the addition of 2 M aqueous HCl (10 vol.) and extracted with EtOAc (2×10 vol.). The combined organics were washed with 1 M aqueous NaHCO₃ (10 vol.), dried over Na₂SO₄ and concentrated under reduced pressure. The crude material was purified by column chromatography (25 g/g loading) with an EtOAc in Heptane gradient. N-Boc-Val-Dil-Dap-OBn was isolated as a clear oil (72%).

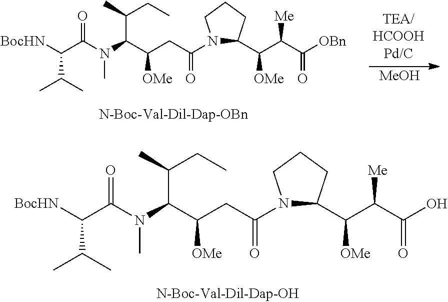 [SMILES: "MeOH" ; CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)OC(C)(C)C)C(C)C ; CC[C@H](C)[C@@H]([C@@H](CC(=O)N1CCC[C@H]1[C@H](OC)[C@@H](C)C(=O)O)OC)N(C)C(=O)[C@@H](NC(=O)OC(C)(C)C)C(C)C]

N-Boc-Val-Dil-Dap-OBn was dissolved MeOH (10 vol.) and 10% Palladium on activated carbon paste type 487-10R487 (0.1 eq.) was charged. Triethylamine (10 eq.) and formic acid (9 eq.) were added and the reaction stirred at RT for 48 h. Celite (1 wt.) was added and the reaction filtered through a celite pad rinsing with MeOH. The filtrates were concentrated and purified by column chromatography (25 g/g loading) with an EtOAc in Heptane gradient with 0.1% AcOH additive. N-Boc-Val-Dil-Dap-OH was isolated in 95% yield.

### Example 6. Use of Universal Dolastatin Core in the Preparation of Monomethylauristatin E (MMAE)

### A. Preparation of Val-Dil-Dap-(1S,2R)-(+)-norephedrine

A solution of N-Boc-Val-Dil-Dap-OH.DCHA (Example 4) (1 g, 1 equiv.) in iPrOAc (10 vol.) was washed with 4 M aqueous H₃PO₄ (2×2 vol.) then DI Water (2 vol.) and the organic layer dried with Na₂SO₄, filtered and concentrated under reduced pressure. To the residue was added iPrOAc (10 vol.), (1S,2R)-(+)-norephedrine (1.2 equiv.), NMI (1 equiv.), DIPEA (1.5 equiv.) and T3P (50% in EtOAc, 2 equiv.). After stirring at RT for 2 h the reaction was quenched by the addition of 2 M aqueous HCl (10 vol.) and the layers partitioned. The organic layer was washed with 2 M aqueous HCl (10 vol.) then 20% aqueous Na₂CO₃ (10 vol.), dried over Na₂SO₄, filtered and concentrated under reduced pressure. The residue was dissolved in toluene (10 vol.) and 3 M HCl in CPME (5 equiv.). After stirring at RT for 72 h the reaction was quenched by the addition of 1 M aqueous NaHCO₃ to pH 9 and the layers partitioned. The aqueous layer was extracted with iPrOAc (3×10 vol.) and the combined organic layers dried over Na₂SO₄, filtered and concentrated to afford Val-Dil-Dap-(1S,2R)-(+)-norephedrine (650 mg, 80%) which can be utilized in subsequent steps without further purification.

### B. Preparation of N-Boc-MMAE

To a stirred solution of Val-Dil-Dap-(1S,2R)-(+)-norephedrine (100 mg, 1 equiv.) in DMF (10 vol.) was charged HATU (1.5 equiv.), N-Boc-N-Me-Val-OH (1.5 equiv.), and DIPEA (2.5 equiv.). After stirring at RT for 16 h the reaction was quenched by the addition of 2 M aqueous HCl (10 vol.). EtOAc (10 vol.) was charged and the layers partitioned. The aqueous layer was extracted with EtOAc (2×10 vol.) and the combined organic layers washed with 1 M aqueous NaHCO₃ (10 vol.). The organic layer was dried over Na₂SO₄, filtered and concentrated under reduced pressure. The residue was purified by silica gel column chromatography (Sfär HC 10 g) eluting with 80:20 EtOAc/Heptane to 80:20 EtOAc/EtOH gradient. Product containing fractions were combined and concentrated under reduced pressure to afford N-Boc-MMAE (130 mg, 96%) as a colorless oil.

### C. Preparation of MMAE

To a stirred solution of Boc-MMAE (130 mg, 1 equiv.) in toluene (20 vol.) was added 4 M HCl in dioxane (5 equiv.). After stirring at RT for 16 h the reaction was quenched by the addition of 1 M aqueous NaHCO₃ to pH 9 and the layers partitioned. The aqueous layer was extracted with EtOAc (3×10 vol.) and the combined organic layers dried over Na₂SO₄, filtered and concentrated to afford crude MMAE. The residue was purified by silica gel column chromatography (Sfär HC 10 g) eluting with 80:20 EtOAc/Heptane to 60/40 EtOAc/EtOH gradient. Product containing fractions were combined and concentrated under reduced pressure to afford MMAE (100 mg, 80%) as a colorless oil. A portion of the purified material was isolated by preparative rp-HPLC (0.05% formic acid in water and acetonitrile, YMC PackPro C18, 250×20 mm, 10 μm). The structure was verified by ¹H NMR (spectrum consistent with literature) and high-resolution mass spectrometry (ESI, m/z=718.5136; calc. [M+H]⁺ 718.5113).

### Example 7. Preparation of vcMMAE from MMAE

To a stirred solution of MMAE (36 mg, 1 equiv.) in DMF (10 vol.) was added mc-Val-Cit-PABC-PNP (1.1 equiv.), HOPO (1.1 equiv.) and 2,6-lutidine (2 vol.). After stirring overnight at RT the material was purified by preparative rp-HPLC (0.05% formic acid in water and acetonitrile, YMC PackPro C18, 250×20 mm, 10 μm). The positive fractions were lyophilized to isolate vcMMAE (65 mg, 98%) and the structure was verified by ¹H NMR (spectrum consistent with literature) and high-resolution mass spectrometry (ESI, m/z=1316.7820; calc. [M+H]⁺ 1316.7864)

### Example 8. Preparation of vcMeVal-OH

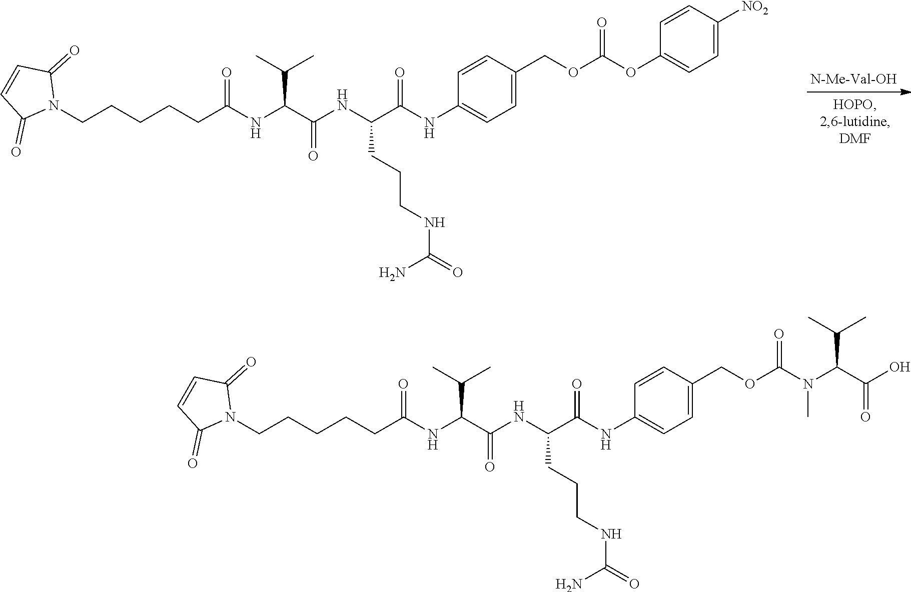 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](CCCNC(N)=O)C(=O)Nc1ccc(COC(=O)N(C)[C@H](C(=O)O)C(C)C)cc1]

To a scintillation vial were added N-Methyl-L-valine (6.2 equiv.) and mc-Val-Cit-PABC-PNP (500 mg, 1.0 equiv.). The vial was purged with nitrogen (×3), and the solids were suspended in 2,6-lutidine (4.0 vol.) and DMF (4.0 vol.). Solid HOPO (1.2 equiv.) was added in one portion, the vessel was sealed, and the reaction was stirred vigorously for 48 h. The reaction was poured into MTBE (200 vol.), and the resulting mixture was vacuum filtered (washing with MTBE) to deliver a grey solid. The solid was solubilized in minimal AcOH (4.0 vol.), and the resulting solution was chromatographically purified on silica gel (5% MeOH in CH₂Cl₂ to 20%) to provide mc-Val-Cit-PAB-N-Me-Val-OH as a pale-yellow residue (200 mg).

### Example 9. Preparation of vcMMAE from Val-Dil-Dap-(1S,2R)-(+)-norephedrine

To a stirred solution of Val-Dil-Dap-(1S,2R)-(+)-norephedrine (50 mg, 1 equiv.) in DMF (10 vol.) was added mc-Val-Cit-PAB-N-Me-Val-OH (1.5 equiv.), HATU (1.5 equiv.) and 2,6-lutidine (10 vol.). The reaction was stirred O/N and purified by preparative rp-HPLC (0.05% formic acid in water and acetonitrile, YMC PackPro C18, 250×20 mm, 10 μm). The compound was isolated (20 mg, 20%) and the structure was verified by HPLC (retention time matched previous vcMMAE material) and high-resolution mass spectrometry (ESI, m/z=1316.7766; calc. [M+H]⁺ 1316.7864).

### Example 10. Preparation of Val-Dil-Dap-Phe-OMe from Universal Dolastatin Core

A solution of N-Boc-Val-Dil-Dap-OH.DCHA (Example 4) (0.525 g, 1 equiv.) in iPrOAc (10 vol.) was washed with 4 M aqueous H₃PO₄ (2×2 vol.) then DI Water (2 vol.) and the organic layer dried with Na₂SO₄, filtered and concentrated under reduced pressure. To the residue was added iPrOAc (10 vol.), L-phenylalanine methyl ester hydrochloride (1.1 equiv.), NMI (1 equiv.), DIPEA (2.5 equiv.) and T3P (50% in EtOAc, 2 equiv.). After stirring at RT for O/N the reaction was quenched by the addition of 2 M aqueous HCl (10 vol.) and the layers partitioned. The organic layer was washed with 2 M aqueous HCl (10 vol.) then 20% aqueous Na₂CO₃ (10 vol.) dried over Na₂SO₄, filtered and concentrated under reduced pressure. The residue was dissolved in toluene (5 vol.), 1,4-dioxane (5 vol.) and 3 M HCl in CPME (5 equiv.). After stirring at RT for 16 h the reaction was quenched by the addition of 1 M aqueous NaHCO₃ to pH 9 and the layers partitioned. The aqueous layer was extracted with iPrOAc (3×10 vol.) and the combined organic layers dried over Na₂SO₄, filtered and concentrated to afford Val-Dil-Dap-Phe-OMe (350 mg, 80%) which can be utilized without further purification.

### Example 11. Preparation of Monomethylauristatin F (MMAF)

### A. Preparation of Boc-MMAF-OMe

To a stirred solution of Val-Dil-Dap-Phe-OMe (100 mg, 1 equiv.) in DMF (10 vol.) was charged HATU (1.1 equiv.), N-Me-Val-OH (1.1 equiv.) and 2,6-lutidine (10 vol.) After stirring at RT for 2 h the reaction was quenched by the addition of 2 M aqueous HCl (10 vol.). EtOAc (10 vol.) was added and the layers partitioned. The aqueous layer was extracted with EtOAc (2×10 vol.) and the combined organic layers dried over Na₂SO₄, filtered and concentrated under reduced pressure. The residue was purified by silica gel column chromatography (Sfär HC 10 g) eluting with 80:20 EtOAc/Heptane to 80:20 EtOAc/EtOH gradient. Concentration of positive fractions gave Boc-MMAF-OMe (124 mg, 93%) as a colorless residue.

### B. Preparation of MMAF

To a stirred solution of Boc-MMAF-OMe (124 mg, 1 equiv.) in toluene (8 vol.) and 1,4-dioxane (8 vol.) was added 3 M HCl in CPME (16 vol.). The reaction was stirred at RT for 16 h and quenched by the addition of 20% aqueous Na₂CO₃ to pH 11. The mixture was extracted with iPrOAc (3×10 vol.), dried over Na₂SO₄, filtered and concentrated under reduced pressure. The residue was dissolved in 4 M aqueous HCl (20 vol.) and AcOH (20 vol.) and stirred at RT for 24 h. The reaction was purified by preparative rp-HPLC (0.05% formic acid in water and acetonitrile, Phenomenex Kinetex F5, 150×21.2 mm, 5 μm). The highest purity fractions were combined and lyophilized to yield MMAF (7 mg, 5%). The structure was verified by ¹NMR (spectrum consistent with literature) and high-resolution mass spectrometry (ESI, m/z=732.4901; calc. [M+H]⁺ 732.4906).

All examples provided herein are exemplary in nature and are not meant to limit the scope of the invention as defined by the claims.

1. A method for making a dolastatin, auristatin or related compounds comprising the steps of:
providing a compound of Formula I, or salt thereof, 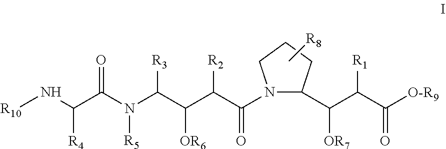
wherein
R₁, R₂, R₃, R₄ R₅ and R₈ are each individually selected from the group consisting of H, C₁-C₆ alkyl, C₁-C₆ substituted alkyl, —OR₁₁, —NR₁₁R₁₂, —SR₁₁ and halo,
R₁₁ and R₁₂ are individually selected from the group consisting of H, C₁-C₆ alkyl,
R₆ and R₇ are each individually H or C₁-C₄ alkyl,
R₉ is H or an acid protecting group, and
R₁₀ is H or an amino protecting group;
if R₉ is an acid protecting group, deprotecting the C-terminal carboxylic acid group,
reacting the C-terminal carboxylic acid group with an amine (A) to form an amide bond;
if R₁₀ is an amino protecting group, deprotecting the N-terminal amine,
reacting the N-terminal amine with a carboxylic acid (CA) to form an amide bond.

2. The method of claim 1 wherein the steps of
if R₉ is an acid protecting group, deprotecting the C-terminal carboxylic acid group,
reacting the C-terminal carboxylic acid group with an amine (A) to form an amide bond;
are performed before the steps of
if R₁₀ is an amino protecting group, deprotecting the N-terminal amine,
reacting the N-terminal amine with a carboxylic acid (CA) to form an amide bond.

3. The method of claim 1 wherein the steps of
if R₁₀ is an amino protecting group, deprotecting the N-terminal amine,
reacting the N-terminal amine with a carboxylic acid (CA) to form an amide bond
are performed before the steps of
if R₉ is an acid protecting group, deprotecting the C-terminal carboxylic acid group,
reacting the C-terminal carboxylic acid group with an amine (A) to form an amide bond.

4. The method of claim 1 wherein the amine (A) is selected from the group consisting of alkylamines, alkanolamines, arylalkanolamines, amino acids, amino acid derivatives, peptides and peptide derivatives,
wherein the amine (A) may have one or more substituents, and
wherein the amine (A) may have a protecting group.

5. The method of claim 4 wherein the amine (A) is selected from the group consisting of phenylalanine, phenylalanine derivatives, substituted phenylalanine, substituted phenylalanine derivatives, tryptophan, tryptophan derivatives, substituted tryptophan, substituted tryptophan derivatives, phenylpropanolamine, protected phenylpropanolamine, substituted phenylpropanolamine, protected substituted phenylpropanolamine, dolaphenine and protected dolaphenine, substituted dolaphenine, protected substituted dolaphenine, dolaphenine derivatives, protected dolaphenine derivatives, substituted dolaphenine derivatives, protected dolaphenine derivatives.

6. The method of claim 1 wherein the carboxylic acid (CA) is selected from the group consisting of amino acids, amino acid derivatives, peptides and peptide derivatives,
wherein the carboxylic acid (CA) may have one or more substituents, and
wherein the carboxylic acid (CA) may have a protecting group.

7. The method of claim 6 wherein the carboxylic acid (CA) is selected from the group consisting of valine, protected valine, substituted valine, protected substituted valine, valine derivatives, protected valine derivatives, substituted valine derivatives, protected substituted valine derivatives, alanine, protected alanine, substituted alanine, protected substituted alanine, alanine derivatives, protected alanine derivatives, substituted alanine derivatives and protected substituted alanine derivatives.

8. The method of claim 1 wherein the carboxylic acid (CA) comprises one or more of a spacer, a linker and an attachment group.

9. The method of claim 1, wherein the compound of Formula I is

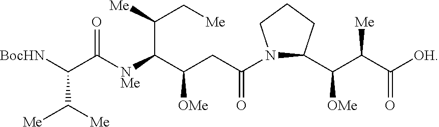

10. An isolated salt of Formula II: 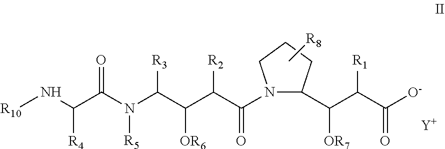
wherein
R₁, R₂, R₃, R₄ R₅and R₈ are each individually selected from the group consisting of H, C₁-C₆ alkyl, C₁-C₆ substituted alkyl, —OR₁₁, —NR₁₁R₁₂, —SR₁₁ and halo,
R₁₁ and R₁₂ are individually selected from the group consisting of H and C₁-C₆ alkyl,
R₆ and R₇ are each individually H or C₁-C₄ alkyl,
R₁₀ is H or an amino protecting group; and
Y⁺ is counterion.

11. The isolated salt of claim 10, wherein
R₁, R₂, R₃, R₄ R₅and R₈ are each individually selected from the group consisting of H, methyl, ethyl, n-propyl, iso-propyl, n-butyl, sec-butyl, tert-butyl, and iso-butyl;
R₆ and R₇ are each individually H or methyl,
R₁₀ is H or tert-butoxycarbonyl (Boc), and
Y⁺ is an ammonium ion of the formula N⁺HR₁₃R₁₄R₁₅ wherein R₁₃ is selected from the group consisting of optionally substituted C₁-C₈ alkyl and optionally substituted C₃-C₈ cycloalkyl; R₁₄ and R₁₅ are independently selected from the group consisting of H, optionally substituted C₁-C₈ alkyl and optionally substituted C₃-C₈ cycloalkyl; wherein each optional substituent, if present, is selected from the group consisting of alkyl and aryl.

12. The isolated salt of claim 11 wherein Y⁺ is selected from the group consisting diethylammonium ion, dibutylammonium ion, dicyclohexylammonium ion, methylcyclohexylammonium ion and methylbenzylammonium ion.

13. The isolated salt of claim 12, wherein the salt is

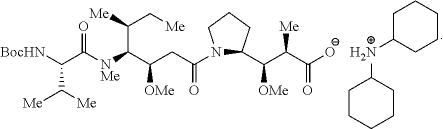

14. A compound of Formula III: 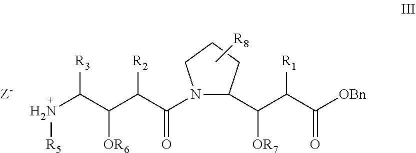
wherein
R₁, R₂, R₃, R₅ and R₈ are each individually selected from the group consisting of H, C₁-C₆ alkyl, C₁-C₆ substituted alkyl, —OR₁₁, —NR₁₁R₁₂, —SR₁₁ and halo,
R₁₁ and R₁₂ are individually selected from the group consisting of H and C₁-C₆ alkyl,
R₆ and R₇ are each individually selected from the group consisting of H or C₁-C₄ alkyl, and
Z⁻ is a counterion.

15. The compound of claim 14, wherein
R₁, R₂, R₃, R₅ and R₈ are each individually selected from the group consisting of H, methyl, ethyl, n-propyl, isopropyl, n-butyl, sec-butyl, iso-butyl, and tert-butyl,
R₆ and R₇ are each individually selected from the group consisting of H and methyl, and
Z⁻ is selected from the group consisting of halide, sulfate, hydrogen sulfate, phosphate, hydrogen phosphate, dihydrogen phosphate, mesylate, tosylate, benzene sulfonate, ethylsulfonate, nitrate, formate, acetate, trifluoroacetate, oxalate, and citrate.

16. The compound of claim 15, wherein the compound is:

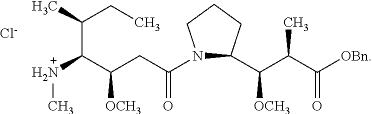 [SMILES: [Cl-] ; CC[C@H](C)[C@H]([NH2+]C)[C@@H](CC(=O)N1CCC[C@H]1[C@H](OC)[C@@H](C)C(=O)OCc1ccccc1)OC]

17. A method of coupling an amino acid to a compound of Formula III 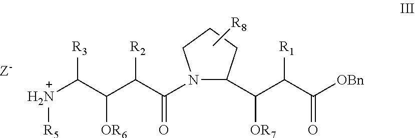
wherein
R₁, R₂, R₃, R₅ and R₈ are each individually selected from the group consisting of H, C₁-C₆ alkyl, C₁-C₆ substituted alkyl, —OR₁₁, —NR₁₁R₁₂, —SR₁₁ and halo,
R₁₁ and R₁₂ are individually selected from the group consisting of H and C₁-C₆ alkyl,
R₆ and R₇ are each individually selected from the group consisting of H or C₁-C₄ alkyl, and
Z⁻ is a counterion
comprising the steps of
contacting the compound of Formula III with an aqueous base to remove the counterion, and
contacting the compound of Formula III with an N-protected amino acid N-carboxyanhydride to yield a compound of Formula IV: 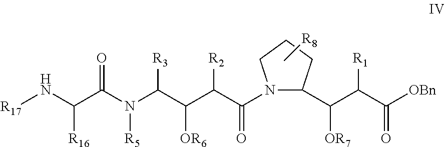
wherein
R₁, R₂, R₃, R₅, R₆, R₇ and R₈ are defined as above,
R₁₆ is an amino acid side chain, and
R₁₇ is a protecting group.

18. The method of claim 17 wherein
R₁, R₂, R₃, R₅ and R₈ are each individually selected from the group consisting of H, methyl, ethyl, n-propyl, isopropyl, n-butyl, sec-butyl, iso-butyl, and tert-butyl,
R₆ and R₇ are each individually selected from the group consisting of H, methyl, ethyl, n-propyl, isopropyl, n-butyl, sec-butyl, iso-butyl, and tert-butyl,
Z⁻ is selected from the group consisting of halide, sulfate, hydrogen sulfate, phosphate, hydrogen phosphate, dihydrogen phosphate, mesylate, tosylate, benzene sulfonate, ethylsulfonate, nitrate, formate, acetate, trifluoroacetate, oxalate, and citrate;
the aqueous base is selected from the group consisting of Na₂CO₃, NaHCO₃, NaOH, Na₂HPO₄, and Na₃PO₄,
the N-protected amino acid N-carboxyanhydride is a Boc-protected amino acid N-carboxyanhydride.

19. The method of claim 18 wherein the compound of Formula III is 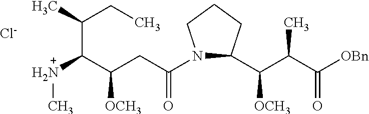 [SMILES: [Cl-] ; CC[C@H](C)[C@H]([NH2+]C)[C@@H](CC(=O)N1CCC[C@H]1[C@H](OC)[C@@H](C)C(=O)OCc1ccccc1)OC]
the aqueous base is Na₂CO₃,
the N-protected amino acid N-carboxyanhydride is Boc-Val-NCA, and
the compound of Formula IV is:

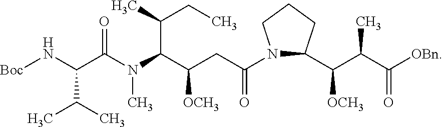 [SMILES: CC[C@H](C)[C@@H]([C@@H](CC(=O)N1CCC[C@H]1[C@H](OC)[C@@H](C)C(=O)OCc1ccccc1)OC)N(C)C(=O)[C@@H](NC(=O)OC(C)(C)C)C(C)C]

20. A method for purifying a crude hydroxy acid of Formula V without using column chromatography, the method comprising the steps of
providing a crude hydroxy acid of Formula V 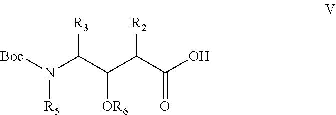
wherein
R₂, R₃, and R₅ are each individually selected from the group consisting of H, C₁-C₆ alkyl, C₁-C₆ substituted alkyl, —OR₁₁, —NR₁₁R₁₂, —SR₁₁ and halo; R₁₁ and R₁₂ are individually selected from the group consisting of H, C₁-C₆ alkyl, and
R₆ is selected from the group consisting of H and C₁-C₄ alkyl;
dissolving crude hydroxy acid in a soluble solvent,
adding an insoluble solvent,
initiating crystallization, and
allowing the crystallization to complete, yielding a purified hydroxy acid of Formula V.

21. The method of claim 20, wherein the hydroxy acid of Formula V is 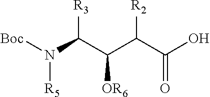
wherein
R₂, R₃ and R₅ are each individually selected from the group consisting of H, methyl, ethyl, n-propyl, isopropyl, n-butyl, sec-butyl, iso-butyl, and tert-butyl,
R₆ is selected from the group consisting of H and methyl,
the soluble solvent comprises tert-butyl methyl ether (MTBE),
the insoluble solvent comprises heptane, and
crystallization is initiated by seeding, by heat cycling or by a combination thereof.

22. The method of claim 21 wherein the hydroxy acid of Formula V is

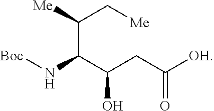 [SMILES: CC[C@H](C)[C@H](NC(=O)OC(C)(C)C)[C@H](O)CC(=O)O]

23. The method of claim 20, further comprising the step of isolating the purified hydroxy acid of Formula V.

24. A compound of Formula VI: 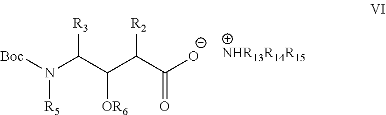
wherein
R₂, R₃, and R₅ are each individually selected from the group consisting of H, C₁-C₆ alkyl, C₁-C₆ substituted alkyl, —OR₁₁, —NR₁₁R₁₂, —SR₁₁ and halo; R₁₁ and R₁₂ are individually selected from the group consisting of H, C₁-C₆ alkyl, and
R₆ is selected from the group consisting of H and C₁-C₄ alkyl; and
R₁₃ is selected from the group consisting of optionally substituted C₁-C₈ alkyl and optionally substituted C₃-C₈ cycloalkyl; R₁₄ and R₁₅ are independently selected from the group consisting of H, optionally substituted C₁-C₈ alkyl and optionally substituted C₃-C₈ cycloalkyl; wherein each optional substituent, if present, is selected from the group consisting of alkyl and aryl.

25. The compound of claim 24, wherein the compound of Formula VI is 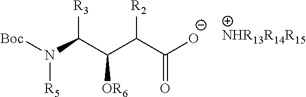
wherein
R₂, R₃ and R₅ are each individually selected from the group consisting of H, methyl, ethyl, n-propyl, isopropyl, n-butyl, sec-butyl, iso-butyl, and tert-butyl, and
R₆ is selected from the group consisting of H and methyl; and
NHR₁₃R₁₄R₁₅ is selected from the group consisting of selected from the group consisting diethylammonium ion, dibutylammonium ion, dicyclohexylammonium ion, methylcyclohexylammonium ion and methylbenzylammonium ion.

26. The compound of claim 25, wherein the compound of Formula VI is:

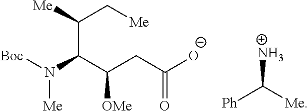 [SMILES: CC[C@H](C)[C@@H]([C@@H](CC(=O)[O-])OC)N(C)C(=O)OC(C)(C)C ; C[C@H]([NH3+])c1ccccc1]

27. The compound of claim 24 wherein the compound of Formula VI is a solid.

28. A compound of Formula VII 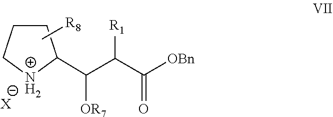
wherein
R₁ and R₈ are individually selected from the group consisting of H, C₁-C₆ alkyl, C₁-C₆ substituted alkyl, —OR₁₁, —NR₁₁R₁₂, —SR₁₁ and halo; R₁₁ and R₁₂ are individually selected from the group consisting of H, C₁-C₆ alkyl;
R₇ is selected from the group consisting of H or C₁-C₄ alkyl; and
X⁻ is selected from the group consisting of halide, sulfate, hydrogen sulfate, phosphate, hydrogen phosphate, dihydrogen phosphate, mesylate, tosylate, benzene sulfonate, ethylsulfonate, nitrate, formate, acetate, oxalate, and citrate.

29. The compound of claim 28 wherein the compound of Formula VII is 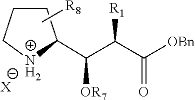
wherein
R₁ and R₈ are individually selected from the group consisting of H, methyl, ethyl, n-propyl, isopropyl, n-butyl, sec-butyl, iso-butyl, and tert-butyl,
R₇ is selected from H and methyl; and
X⁻ is a halide.

30. The compound of claim 29 wherein the compound of Formula VII is

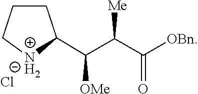 [SMILES: CO[C@@H]([C@@H]1CCC[NH2+]1)[C@@H](C)C(=O)OCc1ccccc1 ; [Cl-]]

31. The compound of claim 30 wherein the compound of Formula VII is a solid.

32. A compound of Formula VIII:

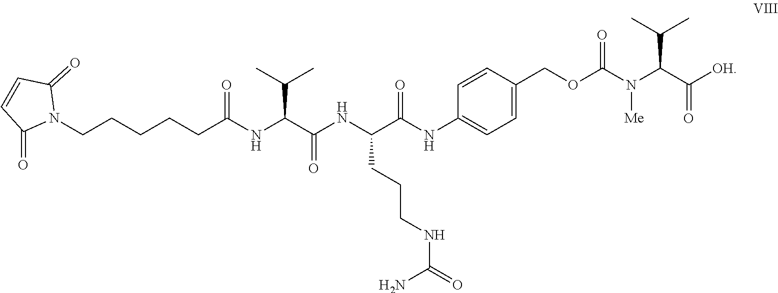 [SMILES: CC(C)[C@H](NC(=O)CCCCCN1C(=O)C=CC1=O)C(=O)N[C@@H](CCCNC(N)=O)C(=O)Nc1ccc(COC(=O)N(C)[C@H](C(=O)O)C(C)C)cc1]

33. The method of claim 7 wherein the carboxylic acid (CA) comprises one or more of a spacer, a linker and an attachment group.

34. The method of claim 1, with the proviso that when R₁, R₅, R₆ and R₇ are methyl, R₂, R₈ and R₉ are H, R₃ is sec-butyl, R₄ is isopropyl, R₁₀ is a tert-butoxycarbonyl (Boc) protecting group, and amine (A) is a salt of phenylalanine, then carboxylic acid (CA) is not protected substituted valine.

35. The method of claim 1, with the proviso that when R₁, R₅, R₆ and R₇ are methyl, R₂, R₈ and R₉ are H, R₃ is sec-butyl, R₄ is isopropyl, R₁₀ is a tert-butoxycarbonyl (Boc) protecting group and carboxylic acid (CA) is protected substituted valine, then amine (A) is not a salt of phenylalanine.
